SEQUENCE LISTING (23 sequences)

SEQ ID NO: 1 - DNA, 20 bp, Artificial Sequence
  Description of Artificial Sequence primer for RT-PCR
aatggtcgaggaccagatgg

SEQ ID NO: 2 - DNA, 20 bp, Artificial Sequence
  Description of Artificial Sequence primer for RT-PCR
ttcaggagcacaacagcagc

SEQ ID NO: 3 - DNA, 18 bp, Artificial Sequence
  Description of Artificial Sequence primer for RT-PCR
tcaccaccatggagaagg

SEQ ID NO: 4 - DNA, 20 bp, Artificial Sequence
  Description of Artificial Sequence primer for RT-PCR
caaagttgtcatggatgacc

SEQ ID NO: 5 - DNA, 23 bp, Artificial Sequence
  Description of Artificial Sequence oligonucleotide
gctgtctcaacggtggtacatgc

SEQ ID NO: 6 - DNA, 23 bp, Artificial Sequence
  Description of Artificial Sequence oligonucleotide
gcatgtaccaccgttgagacagc

SEQ ID NO: 7 - DNA, 23 bp, Artificial Sequence
  Description of Artificial Sequence oligonucleotide
cctggaagaactgcacgcagtct

SEQ ID NO: 8 - DNA, 23 bp, Artificial Sequence
  Description of Artificial Sequence oligonucleotide
agactgcgtgcagttcttccagg

SEQ ID NO: 9 - DNA, 23 bp, Artificial Sequence
  Description of Artificial Sequence oligonucleotide
ggaccttcttgacgtgcgtcaga

SEQ ID NO: 10 - DNA, 23 bp, Artificial Sequence
  Description of Artificial Sequence oligonucleotide
cagcttgcacaaccagacagacc

SEQ ID NO: 11 - DNA, 23 bp, Artificial Sequence
  Description of Artificial Sequence oligonucleotide
ggtctgtctggttgtgcaagctg

SEQ ID NO: 12 - DNA, 23 bp, Artificial Sequence
  Description of Artificial Sequence oligonucleotide
tgcacggttctggttgcgtgtga

SEQ ID NO: 13 - DNA, 29 bp, Artificial Sequence
  Description of Artificial Sequence PCR primer
ttactcgaggcagctggcgagcaggcatg

SEQ ID NO: 14 - DNA, 29 bp, Artificial Sequence
  Description of Artificial Sequence PCR primer
ttagctagccggacattcgcagtagaagg

SEQ ID NO: 15 - DNA, 23 bp, Artificial Sequence
  Description of Artificial Sequence oligonucleotide
gcaggtacgagcgtcattctcac

SEQ ID NO: 16 - DNA, 23 bp, Artificial Sequence
  Description of Artificial Sequence oligonucleotide
gtgagaatgacgctcgtacctgc

SEQ ID NO: 17 - DNA, 23 bp, Artificial Sequence
  Description of Artificial Sequence oligonucleotide
cactgacgtgcatccttgagacg

SEQ ID NO: 18 - DNA, 23 bp, Artificial Sequence
  Description of Artificial Sequence oligonucleotide
agactgcgtgcagttcttccagg

SEQ ID NO: 19 - DNA, 23 bp, Artificial Sequence
  Description of Artificial Sequence oligonucleotide
cctggaagaactgcacgcagtct

SEQ ID NO: 20 - DNA, 23 bp, Artificial Sequence
  Description of Artificial Sequence oligonucleotide
gtcggtatgtactgcgcgtacca

SEQ ID NO: 21 - DNA, 23 bp, Artificial Sequence
  Description of Artificial Sequence oligonucleotide
cctggtagatgaagtcggagatg

SEQ ID NO: 22 - DNA, 23 bp, Artificial Sequence
  Description of Artificial Sequence oligonucleotide
catctccgacttcatctaccagg

SEQ ID NO: 23 - DNA, 23 bp, Artificial Sequence
  Description of Artificial Sequence oligonucleotide
gtatcggacgcggttagaatgga

(19) United States
(12) Patent Application Publication
(10) Pub. No.: US 2005/0187179 A1
(43) Pub. Date: Aug. 25, 2005

(54) Apoptosis inducing agents and methods

(75) Inventors: Lucio Miele, West Chicago, IL (US); Leslie S. Shields, Encinitas, CA (US); Chana Fuchs, Chevy Chase, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignees: The Government of the United States of America as represented by the; Secretary of the Department of Health and Human Services

(21) Appl. No.: 11/069,208

(22) Filed: Feb. 28, 2005

Related U.S. Application Data
(63) Continuation of application No. 09/806,440, filed on Mar. 30, 2001.
(60) Provisional application No. 60/124,119, filed on Mar. 12, 1999.
(60) Provisional application No. 60/102,816, filed on Oct. 2, 1998.

(51) Int. Cl. A61K 48/00

(52) U.S. Cl. 514/44; 514/167; 514/616; 514/12; 514/169

(57) ABSTRACT

Methods and compositions are disclosed for inducing differentiation and apoptosis in cells that overexpress Notch proteins. A cell fate determining function of Notch is specifically disrupted at a time when the cell is undergoing differentiation, which causes the cell to undergo apoptosis. The invention includes therapies for tumors that overexpress a Notch protein (such as Notch-1) by inducing differentiation of the cells in the tumor with a differentiation inducing agent, such as HMBA, in combination with an agent that disrupts the function of the Notch protein. At a time during which differentiation has been promoted, and the cell is susceptible to interference with the anti-apoptosis effect of Notch, the function of the Notch protein is disrupted. Disruption of Notch function can be achieved, for example, by a differentiation inducing agent, such as HMBA, combined with antibodies that specifically bind to Notch and inactivate it, for example a monoclonal antibody that recognizes Notch-1 EGF-like repeats 11 and 12, such as monoclonal antibodies A6, C11 or F3. Disruption of Notch function can also be achieved by the expression of antisense oligonucleotides that specifically interfere with expression of the Notch protein on the cell, alone or in combination with antineoplastic agents.

### FIELD OF THE INVENTION

This invention concerns compositions and methods for stimulating/increasing cell differentiation and inducing apoptosis, and is particularly related to the treatment of tumors which have increased Notch expression.

### BACKGROUND OF THE INVENTION

The Notch gene belongs to the family of epidermal growth factor (EGF)-like homeotic genes, which encode transmembrane proteins with a variable number of cysteine-rich EGF-like repeats in the extracellular region. All four Notch genes (Notch 1-4), which have been described in mammals, have been implicated in the differentiation of the nervous system and other structures (Lardelli et al., Int. J. Dev. Biol. 1995, 39:769-80; Jhappan et al., Genes Dev. 1992, 6:345-55; Robbins et al., J. Virol. 1992, 66:2594-9). The EGF-like proteins Delta and Serrate have been identified as ligands of Notch-1.

Mature Notch proteins are heterodimeric receptors derived from the cleavage of Notch pre-proteins into an extracellular subunit (N^{EC}) containing multiple EGF-like repeats and a transmembrane subunit including the intracellular region (N^{TM}) (Blaumueller et al., Cell 1997, 90:281-91). Notch activation results from the binding of ligands expressed by neighboring cells or soluble ligands (Qi et al., Science 1999, 283:914), and signaling from activated Notch involves networks of transcription regulators (Artavanis-Tsakonas et al., Science 1995, 268:225-32).

Several groups have independently generated antibodies which recognize Notch-1. Kidd et al. (Genes Devel. 1989, 3:1113-29) produced monoclonal antibodies and Johansen et al. (J. Cell Biol. 1989, 109:2427-40) produced polyclonal antibodies which recognize the extracellular portion of Notch, to study its expression in Drosophila. Fehon et al. produced mouse monoclonal antisera against the intracellular domain of Notch and mouse polyclonal antisera against the extracellular domain of Notch to study the interaction between Delta and Notch (Cell 1990, 61:523-34) and to examine Notch expression in Drosophila (J. Cell Biol. 1991, 113:657-69). Hasserjian et al. (Blood 1996, 88:970-6) disclose the generation of anti-Notch antibodies to detect Notch expression on T-cells. Antibodies specific to EGF-like repeats 11 and 12 of Notch have been generated for use in immunoassays (U.S. Pat. No. 5,648,464), for therapeutic and diagnostic methods of cancers in which Notch is overexpressed (U.S. Pat. No. 5,786,158) and to inhibit differentiation of 3T3-L1 cells (Garcés et al., J. Biol. Chem. 1997, 272:29729-34). Zagouras et al. (Proc. Natl. Acad. Sci. USA 1995, 92:6414-8) generated several polyclonal antisera against non-conserved regions of Notch-1, to examine Notch-1 expression in tumor cells.

It has been proposed by Lindsell et al. (Cell 1995, 80:909-17) that activated Notch functions by maintaining the cells in an undifferentiated state. In chicken retina explants, expression of a constitutively activated Notch-1 inhibits differentiation of retinal ganglion cell progenitors, while antisense oligonucleotides increase differentiation towards a neuronal phenotype (Austin et. al, Development 1995, 121:3637-50). However, antisense Notch-1 expression prevents adipocyte differentiation (Garcés et al., J. Biol. Chem. 1997, 272:29729-34). In mice, the downregulation of Notch-1 is required for maturation of cortical thymocytes (Hasserjian et al. Blood 1996, 88:970-6) while its expression coordinates the process of somitogenesis (Conlon et al. Development 1995, 121:153345). In other experimental models, such as CD4/CD8 and α/β versus γ/δ lineage decisions in thymocytes, and in vitro adipocyte differentiation of 3T3-L1 cells, expression of Notch-1 appears to be necessary for proper interpretation of differentiation stimuli (Robey et al., Cell 1996, 87:483-92; Washburn et al., Cell 1997, 88:83343; and Garcés et al., J. Biol. Chem. 1997, 272:29729-34).

Alteration of Notch signaling or expression may contribute to tumorigenesis. Deletions of the extracellular portion of human Notch-1 are associated with about 10% of the cases of T-cell acute lymphoblastic leukemia (Ellison et al., Cell 1991, 66:649-61). Truncated forms of Notch-1 cause T-cell lymphomas when introduced into mouse bone marrow stem cells (Pear et al., J. Exp. Med. 1996, 183:2283-91). Truncated forms of both Notch-1 and Notch-2 have been shown to be oncogenic in rat kidney cells (Capobianco et al., Mol. Cell. Biol. 1997, 17:6265-73). The human Notch-1 gene is in a chromosomal region (9q34) associated with hematopoietic malignancies of lymphoid, myeloid and erythroid lineage (Larson et al., Genomics 1994, 24:253-8). Additionally, strikingly increased expression of Notch-1 has been documented in a number of human tumors including cervical cancer, colon tumors, and lung tumors (Daniel et al., J. Gen. Virol. 1997, 78:1095-101). Increases in both Notch-1 and Notch-2 expression has been observed in pre-neoplastic lesions of the uterine cervix (Zagouras et al., Proc. Natl. Acad. Sci. USA 1995, 92:6414-8).

Many transformed cells retain the capacity to undergo terminal differentiation when treated with pharmacological agents belonging to one of several classes of differentiation-inducing drugs. For example, hybrid polar compounds (which have both polar and apolar regions) can induce differentiation in transformed cells derived from many tissues of all embryonic lineages (Marks et al., Int. J. Henatol. 1996, 63:1-17). The prototype of this class, hexamethylene bisacetamide (HMBA) has been extensively characterized in vitro, and has been clinically tested in patients with myelodysplastic syndrome and acute myeloblastic leukemia (Andreef et al., Blood 1992, 80:2604-9). Unfortunately, this therapy can result in thrombocytopenia. Therefore, the identification of agents which can enhance the effect of HMBA, so that the amount administered could be decreased, would be desirable.

The mechanism of action of HMBA has been studied in detail in murine erythroleukemia (MEL) cell lines, which are retrovirus-transformed hematopoietic precursors which are induced by hybrid polar agents to differentiate along the erythroid lineage. Exposure to HMBA during the G1 phase of the cell cycle causes the following G1 to be prolonged. Thereafter, cell cycle rates return to normal, and at each cycle a fraction of the cells are stochastically committed to terminal differentiation. This G1 lag is necessary, but not sufficient for commitment to terminal differentiation in response to HMBA (Kiyokawa et al., Proc. Natl. Acad. Sci. USA 1993, 90:6746-50). Additional, still unidentified, biochemical events which occur during subsequent cycles are required to trigger terminal differentiation.

PCT publication WO 94/07474 and U.S. Pat. No. 5,786,158 disclosed administration of Notch proteins, sense or antisense nucleic acids, and Notch antibodies, to treat disorders of cell fate or differentiation, including cancer, without specifying how the Notch proteins would affect differentiation. Garcés et al. (J. Biol. Chem. 1997, 272:29729-34) determined that administration of a recombinant protein encompassing EGF-like repeats 11 and 12 of Notch-1, polyclonal antiserum directed against these same repeats, or an antisense nucleotide encompassing an intracellular domain of Notch-1, inhibited the differentiation of 3T3-L1 cells. Combined, these data indicate that agents which disrupt normal Notch-1/ligand interactions produces the same effects as the inhibition of Notch-1 expression by genetic means. That is, they inhibit differentiation. This is in contrast to the administration of HMBA, which induces differentiation.

### SUMMARY OF THE INVENTION

The results disclosed in this application reveal a new mechanism by which to treat cancer cells which overexpress Notch. The unexpected finding that administration of either Notch antisense oligonucleotides or monoclonal antibodies directed to Notch (for example to the EGF-like repeats 11 and 12 of Notch-1), when administered with HMBA, enhanced differentiation to a greater extent than HMBA alone, and provides a novel means to treat tumor cells.

In spite of extensive research concerning Notch proteins, their therapeutic use has not been possible. Although WO 94/07474 and U.S. Pat. No. 5,786,158 indicated that Notch antibodies and Notch antisense oligonucleotides (or other molecules that interfere with the expression or function of Notch) could be therapeutically administered to treat or prevent tumors, it has now been found that administration of either Notch antisense oligonucleotides or monoclonal antibodies directed to the EGF-like repeats 11 and 12 of Notch-1, alone is ineffective as an anti-neoplastic treatment. The present invention has overcome this problem by combining the administration of a cell differentiating agent with a molecule that interferes with the expression or function of a Notch protein (such as the Notch-1 protein). This combination of approaches has unexpectedly been found to induce differentiation in neoplastic cells, even where substantial differentiation would not otherwise be observed, and for the first time provides a useful therapeutic application of this technology.

The method of the present invention therefore includes inducing apoptosis in a target cell by inhibiting a cell fate determining function of a Notch protein in the target cell at a time when the cell is undergoing differentiation. In particular embodiments, the target cell is induced to differentiate and upregulate Notch expression, so that interference with Notch expression or function causes the cell to commit to an apoptotic pathway. Inhibition of Notch expression or interference with its function can include exposing the cell to a Notch protein antisense oligonucleotide that includes at least six nucleotides that comprise a sequence complementary to at least a portion of an RNA transcript of a Notch gene (such as the Notch-1 or Notch-2 gene), and is hybridizable to the RNA transcript. Although the antisense oligonucleotide can be hybridizable to any region of the RNA transcript, particular oligonucleotides that have been found to be useful are antisense oligonucleotides to the Notch-1 EGF repeat region, Lin Notch region, or ankyrin region (shown respectively in SEQ. ID. NOS. 6, 8 or 11). Alternatively, the molecule can be a molecule that antagonizes the function of a Notch protein in the cell, such as an antibody (or a portion containing a binding domain thereof) that specifically binds to Notch.

The method of the present invention also includes inducing apoptosis in a target cell by inhibiting a cell fate determining function of a Notch protein in the target cell at a time when the cell is undergoing differentiation, further comprising treating the target cell with a therapeutically effective amount of another antineoplastic agent at a time that enhances apoptosis in the target cell. The other antineoplastic agent includes for example vinca alkaloids, for example vinblastine, Paclitaxel and vincristine. The antineoplastic agent can also be administered substantially concurruently with the agent administered to inhibit a cell fate determining function of a Notch protein in the target cell at a time when the cell is undergoing differentiation, which induces the target cell to undergo apoptosis. In a further embodiment, a method of inducing apoptosis in a tumor cell that is characterized by increased expression of a Notch protein by administering a therapeutically effective amount of a first antineoplasic agent to a subject having a tumor and interfering with the Notch function or expression in the cells of the tumor, at a time during differentiation when the Notch is required to prevent apoptosis, by administering a molecule that specifically interferes with the Notch function or expression at a time that enhances an effect of the first antineoplastic agent. The first antineoplastic agent which interfers with the Notch function or expression can include a Notch antisense oligonucleotide that specifically blocks expression of the Notch protein or an antibody which specifically binds to the Notch protein and interferes with Notch function. The tumor can be selected from the group consisting of cervical cancer, breast cancer, colon cancer, melanoma, seminoma, lung cancer, and hematopoietic malignancy.

The antibodies of the present invention include those generated against the human Notch-1 EGF-like repeats 11 and 12. These antibodies recognize an extracellular epitope of Notch-1 and stimulate target cell differentiation in the presence of an effective amount of differentiation inducing agent. In particular embodiments, the antibody is a monoclonal antibody, secreted by a hybridoma designated A6 having the A.T.C.C. Accession No. HB12654, a monoclonal antibody, secreted by a hybridoma designated C11 having the A.T.C.C. Accession No. HB12656 and a monoclonal antibody secreted by a hybridoma designated F3 having the A.T.C.C. Accession No. HB12655. In another embodiment, the antibodies are polyclonal antibodies. These monoclonal and polyclonal antibodies enhance the rate of differentiation of a tumor cell that overexpresses Notch-1, when co-administered with a differentiation inducing agent. This treatment leads to eventual apoptosis of the tumor cell. In a particular example of the antibodies, the biologically active human Notch-1 EGF repeats 11 and 12 is not reduced to cleave a disulfide bond.

In a particular embodiment, the target cell is a tumor cell characterized by increased activity or increased expression of a Notch protein, such as a Notch-1 or Notch-2 protein, relative to Notch activity or expression in a same tissue type that is not neoplastic. Examples of such tumor types that overexpress Notch-1 include cervical cancer, breast cancer, colon cancer, melanoma, seminoma, lung cancer, and hematopoietic malignancies, such as erythroid leukemia, myeloid leukemia (such as chronic or acute myelogenous leukemia), neuroblastoma and medulloblastoma. Both Notch-1 and Notch-2 are overexpressed in pre-neoplastic lesions of the uterine cervix. The differentiation inducing agent to which the cell is exposed can be selected from a broad variety of agents, including retinoids, polar compounds, short chain fatty acids, organic acids, Vitamin D derivatives, cyclooxygenase inhibitors, arachidonate metabolism inhibitors, ceramides, diacylglycerol, cyclic nucleotide derivatives, hormones, hormone antagonists, biologic promoters of differentiation, and derivatives of any of these agents. In particular examples, the differentiation inducing agent is a polar hybrid compound, such as hexamethylene bisacetamide (HMBA).

In more particular embodiments, the method includes treating a tumor in a subject by causing apoptosis in tumor cells that expresses Notch protein, and particularly cells that exhibit increased expression of Notch. In this method, differentiation of the tumor cell is induced by administering a differentiation inducing agent to the subject, and interfering with Notch function or expression by administering a molecule that interferes with the Notch function or expression at a time when that function is required to prevent the cell from undergoing apoptosis. The molecule may be an antisense nucleotide, such as an oligonucleotide (up to 100 bases in length) or polynucleotide (which includes nucleotides greater than 200 bases in length) that interferes with expression of the Notch-1 protein. Alternatively, the molecule can be an antibody which binds to the Notch protein (particularly its extracellular receptor portion) and interferes with Notch function.

The invention also includes a pharmaceutical composition that includes a differentiation inducing agent and a molecule that interferes with expression of Notch protein, or a cell fate determining function of the Notch protein, the agent and molecule in combination being present in an effective antineoplastic amount. The molecule may comprise an oligonucleotide having at least six nucleotides from a sequence complementary to at least a portion of an RNA transcript of a Notch gene, such as the Notch-1 gene, and is hybridizable to the RNA transcript thereof. In particular embodiments, the oligonucleotide is the oligonucleotide of SEQ. ID. NOS. 6, 8, or 11, or a subsequence thereof. Alternatively, the molecule may comprise an antibody that specifically binds to Notch (or a portion containing the binding domain), for example a monoclonal antibody directed against Notch-1 EGF-like repeats 11 and 12, that interferes with expression of Notch protein, or a cell fate determining function of the protein, and a pharmaceutically acceptable carrier, with the agent and monoclonal antibody in combination being present in an effective antineoplastic amount. In particular embodiments, the antibody is a monoclonal antibody secreted by a hybridoma designated A6, C11 or F3, having A.T.C.C. Numbers HB12654, HB12656 and HB12655 respectively. In particular embodiments, the differentiation inducing agent may be selected from the group of retinoids, polar compounds, short chain fatty acids, organic acids, Vitamin D derivatives, cyclooxygenase inhibitors, arachinodate metabolism inhibitors, ceramides, diacylglycerol, cyclic nucleotide derivatives, hormones, hormone antagonists, and biologic promoters of differentiation, and derivatives of any of these agents that induce differentiation.

The invention also includes methods for diagnosis and staging of tumor cells which overexpress Notch relative to Notch levels in a same tissue type that is not neoplastic, using an antibody generated against Notch. In a particular embodiment, the antibodies are monoclonal antibodies generated against the human Notch-1 EGF-repeats 11 and 12, that recognizes an extracellular epitope of Notch-1, and that stimulates target cell differentiation in the presence of an effective amount of differentiation inducing agent for immunostaining. In specific embodiments, the antibody is a monoclonal antibody selected from a hybridoma designated A6, C11, or F3. The tumor may be a cervical cancer or the tumor cells are in a Pap smear.

The invention also includes the following hybridomas: A6 having A.T.C.C. Accession No. HB12654; C11 having A.T.C.C. Accession No. HB12656; and F3 having A.T.C.C. Accession No. HB12655.

The foregoing and other objects, features, and advantages of the invention will become more apparent from the following detailed description of a preferred embodiment which proceeds with reference to the accompanying drawings.

### BRIEF DESCRIPTION OF THE DRAWINGS

FIG. 1 is a digital image illustrating protein gels stained with Coomassie used to examine the expression (A) and purification (B-C) of human recombinant Notch-1 EGF-repeats 11 and 12 (rh11-12). a) Time course of recombinant rh11-12 expression following IPTG induction. B) Reducing and C) non-reducing SDS-PAGE analysis of rh11-12 recombinant protein purification. M=markers; l=lysate; a=affinity column pool; s=size exclusion column void volume eluate; p=purified material after size exclusion. Arrows indicate the rh11-12 recombinant protein band.

FIG. 2 is a digital image of western blots illustrating the analysis of polyclonal antibodies generated against rh11-12. A) Polyclonal antibodies to rh11-12 recognize Notch-1 precursor and a major cleavage product (N^{EC}); P=preimmune serum, I=immune serum, R=rh11-12. B-D) A more detailed analysis of human Notch-1 precursor and mature forms in Molt-4 human T-cell leukemia cells by 4% SDS-PAGE to increase the resolution in the high-molecular weight range. Gels were run in the presence of: B) 2-mercaptoethanol; C) DTT; or D) no reducing agents (non-reducing gel). Arrows indicate the rh11-12 recombinant protein band.

FIG. 3 is a digital image of a northern (A) and a western (B) blot illustrating the overexpression of Notch-1 mRNA and protein in transformed cells. J=Jurkat cells, N=negative control; CD4=CD4 cells; CD8=CD8 cells; t=total T-cells; M=Molt-4 cells.

FIG. 4 is a digital image of a western blot illustrating the expression of Notch-1 in human neuroblastoma (SY5Y, DAOY), medulloblastoma (NGP) and Molt-4 (M) cell lines treated with (RA) or without (C) retinoic acid. NGP cells were treated with RA for either two days (2d) or four days (4d). The uppermost arrow indicates Notch-1 pre-protein and the lowermost indicates N^{EC}.

FIG. 5 is a digital image of a western blot illustrating the immunoprecipitation of a Molt-4 cell lysate using two (C11 and A6) of the three monoclonal antibodies, and the polyclonal serum, to capture Notch-1 proteins. Proteins precipitated by the antibodies were detected with the polyclonal Notch-1 antiserum. IP=immunoprecipitation; W=western blotting.

FIG. 6 is a digital image of a western blot illustrating that three mAbs (C11, A6 and F3) immunoprecipitate an N^{EC} band which is available for biotinylation at the cell surface. Proteins precipitated by the various antibodies were detected with the polyclonal Notch-1 antiserum.

FIG. 7 is a digital image showing a section of a human colon adenomatous polyp at 200× magnification, immunostained with C11 (A) or F3 (B) monoclonal antibodies. The negative control is shown in (C).

FIGS. 8A and 8B are digital images of RT-PCR and a western blot, respectively, and c is a line graph, illustrating that HMBA regulates Notch-1 expression in MEL cells. A) Total cellular RNA analyzed by RT-PCR to obtain the total level of Notch-1 and GAPDH mRNA at 4, 8, 24 and 120 hours in the presence (H) or absence (C) of HMBA. B) Digital images of western blots from MEL cells maintained for 4, 24 or 120 hours in the absence (C) or presence (H) of HMBA. Three immunoreactive bands (designated by arrows) are recognized by the Notch-1 antibody. C) Percent differentiation of MEL cells over 120 hours after induction of differentiation with HMBA. At the indicated times, MEL cells were removed and stained with benzidine, which detects differentiated cells.

FIG. 9 is a bar graph showing the effect of Notch-1 mAbs (C11, A6 and F3) on HMBA-induced MEL cell differentiation. Monoclonal antibodies were produced using A) acites or B) a hollow fiber bioreactor, and then purified by protein A affinity chromatography.

FIG. 10 is a dot plot showing the effect of pretreatment with (A) Notch-1 antibody A6 or (B) control antibody IgG2b followed by HMBA treatment on apoptosis.

FIG. 11A is a bar graph illustrating the percent differentiation of MEL cells that were exposed to sense or antisense S-oligonucleotides corresponding to the EGF repeat (EGF), the lin/Notch (LIN) or the ankyrin (ANK) region.

FIG. 11B is a bar graph illustrating the percent of a MEL cell population that is viable or which has undergone apoptosis where the cells have been maintained in the presence of HMBA alone (H) and either a scrambled oligonucleotide (SCR), sense (SEN) or antisense (AS) LIN Notch-1 S-oligonucleotides for 120 hours.

FIGS. 12A and 12B are digital images representing Western blot analysis of Notch-1 protein levels in Notch-1 AS (AS5) or vector-transfected (V5) MEL cells maintained in HMBA for A) one time period or B) several time periods. The Notch-1 extracellular band N^{EC} is shown.

FIG. 13A-C are graphs showing A) a time course of percentage differentiation of MEL cells transfected with vector alone (VECTOR) or with a vector expressing a Notch-1 AS oligonucleotide (ANTISENSE) maintained in HMBA over a period of 120 hours, B and C) the growth kinetics of transfected MEL clones in the B) presence or C) absence of HMBA.

FIGS. 14A-14D are graphs which illustrate percentage apoptosis (A, C) or percentage viability (B, D) for MEL cells transfected with a vector expressing Notch-1 antisense oligonucleotides (ANTISENSE) or vector alone (VECTOR). Cell lines were maintained in the presence (A, B) or absence (C, D) of HMBA for 120 hours.

FIG. 15 is a schematic drawing of the proposed effects of Notch in cell fate determination. In the absence of HMBA, Notch affects the apoptotic threshold during normal growth. During HMBA-induced differentiation essentially all cells undergo a G1 lag followed by progressive recruitment to commitment to terminal differentiation and growth arrest. Committed MEL cells generally undergo 2-5 further rounds of cell division before terminally differentiating. Notch prevents precommitted cells from undergoing apoptosis, thus enabling them to progress through the commitment stage. It is unclear whether Notch affects cell fate decision in committed cells, since it is undetectable 120 hours after HMBA exposure, when most cells are either committed or differentiated.

### SEQUENCE LISTING

SEQ ID NO 1: Sense primer specific for Notch-1, for RT-PCR.

SEQ ID NO 2: Antisense primer specific for Notch-1, for RT-PCR.

SEQ ID NO 3: Sense primer specific for GAPDH, for RT-PCR.

SEQ ID NO 4: Antisense primer specific for GAPDH, for RT-PCR.

SEQ ID NO 5: Sense oligo for the Notch-1 EGF repeat region.

SEQ ID NO 6: Antisense oligo for the Notch-1 EGF repeat region.

SEQ ID NO 7: Sense v for the Lin/Notch region.

SEQ ID NO 8: Antisense oligo for the Lin/Notch region.

SEQ ID NO 9: Scrambled oligo of the Lin/Notch region.

SEQ ID NO 10: Sense oligo for the Notch-1 Ankyrin region.

SEQ ID NO 11: Antisense oligo for the Notch-1 Ankyrin region.

SEQ ID NO 12: Scrambled oligo for the Notch-1 Ankyrin region.

SEQ ID NO 13: Sense PCR primer for Notch-1.

SEQ ID NO 14: Antisense PCR primer for Notch-1.

SEQ ID NO 15: Antisense oligo for the Hu-EGF 34/35 region.

SEQ ID NO 16: Sense oligo for the Hu-EGF 34/35 region.

SEQ ID NO 17: Scrambled oligo for the Hu-EGF 34/35 region.

SEQ ID NO 18: Antisense oligo for the Hu-LIN 12 region.

SEQ ID NO 19: Sense oligo for the Hu-LIN 12 region.

SEQ ID NO 20: Scrambled oligo for the Hu-LIN 12 region.

SEQ ID NO 21: Antisense oligo for the Hu-CDC2 region.

SEQ ID NO 22: Sense oligo for the Hu-CDC2 region.

SEQ ID NO 23: Scrambled oligo for the Hu-CDC2 region.

### DETAILED DESCRIPTION OF SPECIFIC EMBODIMENTS

### Abbreviations and Definitions

AS: Antisense
GAPDH: Glyceraldehyde-3-phosphate dehydrogenase
HMBA: Hexamethylene bisacetamide
IPTG: Isopropyl β-thiogalactopyranoside
KLH: Keyhole limpet hemocyanin
PBS: Phosphate buffered saline
N^{EC}: Notch extracellular subunit
N^{TM}: Notch transmembrane subunit
RA: retinoic acid
RT-PCR: Reverse transcriptase-polymerase chain reaction

Jurkat cells: A human acute T-cell leukemia cell line from American type culture collection (Manassas, Va.). ATCC number TIB-152.

MEL: A mouse erythroleukemia cell line from American Type Culture Collection (Manassas, Va.). ATCC number TIB-55.

Molt-4 cells: A human acute lymphoblastic leukemia cell line from American type culture collection (Manassas, Va.). ATCC number CRL-1582.

Antineoplastic agent: A drug or biologic that inhibits the proliferation of neoplastic cells, for example arresting their growth or causing the regression of a tumor. Includes the vinca alkaloids, for example vinblastine, Paclitaxel, and vincristine.

Differentiation or Differentiation-inducing Agent: An agent that enhances or induces differentiation when exposed to cells. The differentiation agent can be selected from a broad variety of agents, including retinoids, polar compounds, short chain fatty acids, organic acids, Vitamin D derivatives, cyclooxygenase inhibitors, arachidonate metabolism inhibitors, ceramides, diacylglycerol, cyclic nucleotide derivatives, hormones, hormone antagonists, biologic promoters of differentiation, and derivatives of any of these agents. In addition, the differentiation agent can be a polar hybrid compound, such as hexamethylene bisacetamide (HMBA). When added to MEL cells for 48 to 120 hours, these differentiation agents increase differentiation, as determined by the presence of hemoglobin using benzidine staining (see Example 7), 35 to 45% at 120 hr. In other cell types, these differentiation agents may increase differentiation by as little as 20%, or as much as 90%.

Hybridoma: A single-cell cloned cell that secretes a homogenous population of monoclonal antibodies.

Notch antibody: A Notch antibody is one that specifically recognizes one of the Notch proteins, such as Notch 1-4, or any as yet undiscovered Notch. In one embodiment, a Notch antibody is an antibody which recognizes Notch-1 EGF-like repeats 11 and 12, and when added to cells in the presence of a differentiation inducing agent, enhances differentiation. In another embodiment, a Notch antibody is an antibody which recognizes Notch-2 EGF-like repeats 11 and 12, and when added to cells in the presence of a differentiation inducing agent, enhances differentiation. In another embodiment, a Notch antibody is an antibody which recognizes the ligand-binding region of Notch-3. In another embodiment, a Notch antibody is an antibody which recognizes the ligand-binding region of Notch-4. The ligand-binding region is an extracellular domain of Notch. To generate the antibodies, the ligand-binding region, or domains thereof, can be recombinantly expressed, for example in bacteria. The resulting recombinant protein or protein fragment is used to generate antibodies which specifically recognize Notch, and when added to cells in the presence of a differentiation inducing agent, enhances differentiation.

mAb: Monoclonal antibody. An antibody secreted by a hybridoma, which recognizes only one antigen epitope. For generation of monoclonal antibodies, see Examples 4 and 10.

pAb: Polyclonal antibody. A heterogenous population of antibodies which may recognize several different epitopes on a single antigen. For generation of polyclonal antibodies, see Examples 2 and 18.

Notch gene/Notch protein: As used herein, Notch refers to any of the four Notch genes, Notch 1, 2, 3, or 4, or a later identified Notch gene. A Notch protein is the product of one of the Notch genes (Notch 1, 2, 3, 4 or a later identified Notch gene) in vertebrates (such as humans) or invertebrates (such as Drosophilia). The four human Notch genes reside on separate chromosomes, with the Notch-1 gene at chromosome position 9q34 (Ellisen et al., Cell 1991, 66:649-61), while the Notch-2 and Notch-3 genes are located at 1p13-p11 and 19p13.2-p13.1, respectively. The chromosomal location of Notch-4 is not as well characterized. Complete DNA sequences, or cDNA sequences, of each of these human genes may be found in Genbank under accession numbers: M73980 (Notch-1/TAN1); U97669 (Notch-3); and U95299 (Notch-4). DNA and amino acid sequences of Notch-1 have also been disclosed in WO 94/07474. The Notch-2 complete coding sequence is not available in Genbank, although the accession numbers of partial Notch-2 sequences are: X80115; U50549; U77493. The corresponding amino acid sequences can be determined from the DNA sequences, for example as in U.S. Pat. No. 5,648,464. The EGF repeats correspond to amino acid residues 24 to 1449, the ank region corresponds to amino acid residues 1825 to 2087, and the lin region corresponds to amino acid residues 1450 to 1564 of the human Notch-1 preprotein that is not cleaved (Ellisen et al., Cell 1991, 66:649-61). The complete cDNA sequence of mouse Notch-2 is accession number D32210. Amino acid sequences of any of the Notch proteins are known from the disclosed DNA sequences.

Notch therapy: A treatment which results in the inhibition of a cell fate determining function of Notch-1, Notch-2, Notch-3 or Notch-4. This treatment disrupts the function of the Notch protein and can be achieved for example by inhibiting Notch expression, or interfering with its function, or other means. Methods can include exposing the cell to a Notch protein antisense oligonucleotide or to an antibody that recognizes Notch.

Oligo/Oligonucleotide: a linear nucleotide sequence of up to about 100 nucleotide bases in length.

Polynucleotide: a linear nucleotide sequence, including sequences of greater than 100 nucleotide bases in length.

S-oligos: Phosphorothioate oligonucleotides, in which the phosphate group of the phosphodiester backbone of the oligonucleotide has been chemically modified to be a phosphorothioate group, to improve the therapeutic properties of the oligo. This is an example of a chemically modified oligonucleotide, which has been modified to improve its resistance to nucleases, and improve its membrane permeability.

Hybridization: DNA molecules and nucleotide sequences which are derived from the disclosed DNA molecules as described above may also be defined as DNA sequences which hybridize under stringent conditions to the DNA sequences disclosed, or fragments thereof.

Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al. (1989), chapters 9 and 11, herein incorporated by reference. By way of illustration only, a hybridization experiment may be performed by hybridization of a DNA molecule (for example, a variation of the Notch-1 cDNA) to a target DNA molecule (for example, the Notch-1 cDNA itself) which has been electrophoresed in an agarose gel and transferred to a nitrocellulose membrane by Southern blotting, a technique well known in the art and described in Sambrook et al., 1989. Hybridization with a target probe labeled with [³²P]-dCTP is generally carried out in a solution of high ionic strength such as 6×SSC at a temperature that is 20-25° C. below the melting temperature, Tₘ, described below. For such Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal. The term Tₘ represents the temperature above which, under the prevailing ionic conditions, the radiolabeled probe molecule will not hybridize to its target DNA molecule. The Tₘ of such a hybrid molecule may be estimated from the following equation: Tₘ=81.5 C−16.6(log₁₀[Na⁺])+0.41(% G+C)−0.63(% formamide)−(600/l); where l=the length of the hybrid in base pairs.

This equation is valid for concentrations of Na⁺ in the range of 0.01 M to 0.4 M, and it is less accurate for calculations of Tₘ in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Chapter 11 of Sambrook et al., 1989).

The Tₘ of double-stranded DNA decreases by 1-1.5° C. with every 1% decrease in homology. Therefore, for this given example, washing the filter in 0.3×SSC at 59.4-64.4° C. will produce a stringency of hybridization equivalent to 90%; that is, DNA molecules with more than 10% sequence variation relative to the target Notch-1 cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target Notch-1 cDNA molecule will not hybridize. The above example is given entirely by way of theoretical illustration. One skilled in the art will appreciate that other hybridization techniques may be utilized and that variations in experimental conditions will necessitate alternative calculations for stringency.

In an embodiment of the present invention, stringent conditions may be defined as those under which DNA molecules with more than 25%, 15%, 10% or 6% sequence variation (also termed “mismatch”) will not hybridize.

The degeneracy of the genetic code further widens the scope of the present invention as it enables major variations in the nucleotide sequence of a DNA molecule while maintaining the amino acid sequence of the encoded protein. Thus, the nucleotide sequence of the Notch-1 cDNA could be changed without affecting the amino acid composition of the encoded protein or the characteristics of the protein. The genetic code and variations in nucleotide codons for particular amino acids is known. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein also comprehended by this invention.

The antisense oligonucleotides described herein hybridize by hydrogen bonding, which includes Watson-Crick, Hoogsteen or reversed Hoogsteen hydrogen bonding between complementary nucleotide units. For example, adenine and thymine are complementary nucleobases which pair through formation of hydrogen bonds. “Complementary” refers to sequence complementarity between two nucleotide units. For example, if a nucleotide unit at a certain position of an oligonucleotide is capable of hydrogen bonding with a nucleotide unit at the same position of a DNA or RNA molecule, then the oligonucleotides are complementary to each other at that position. The oligonucleotide and the DNA or RNA are complementary to each other when a sufficient number of corresponding positions in each molecule are occupied by nucleotide units which can hydrogen bond with each other.

“Specifically hybridizable” and “complementary” are terms which indicate a sufficient degree of complementarity such that stable and specific binding occurs between the oligonucleotide and the DNA or RNA target. An oligonucleotide need not be 100% complementary to its target DNA sequence to be specifically hybridizable. An oligonucleotide is specifically hybridizable when binding of the oligonucleotide to the target DNA or RNA molecule interferes with the normal function of the target DNA or RNA, and there is a sufficient degree of complementarity to avoid non-specific binding of the oligonucleotide to non-target sequences under conditions in which specific binding is desired, for example under physiological conditions in the case of in vivo assays, or under conditions in which the assays are performed. Such binding is referred to as specific interference with expression of the Notch protein.

Specific binding agent: An agent that binds substantially only to a defined target. As used herein, the term “Notch specific binding agent” includes anti-Notch antibodies and other agents that bind substantially to only a Notch protein, such as Notch-1. The antibodies may be monoclonal or polyclonal antibodies that are specific for Notch-1, and particularly its extracellular domain, and more particularly its EGF-like repeats 11 and 12, as well as immunologically effective portions (“fragments”) thereof. Preferably, the antibodies used in the present invention are monoclonal antibodies (or immunologically effective portions thereof) and may also be humanized monoclonal antibodies (or immunologically effective portions thereof). Immunologically effective portions of monoclonal antibodies include Fab, Fab′, F(ab′)₂, Fabc and Fv portions (for a review, see Better and Horowitz, Methods. Enzymol. 1989, 178:476-96). Anti-Notch peptide antibodies may also be produced using standard procedures described in a number of texts, including “Antibodies, A Laboratory Manual” by Harlow and Lane, Cold Spring Harbor Laboratory (1988).

The determination that a particular agent binds substantially only to the Notch peptide may readily be made by using or adapting routine procedures. One suitable in vitro assay makes use of the Western blotting procedure (described in many standard texts, including “Antibodies, A Laboratory Manual” by Harlow and Lane, 1988). Western blotting may be used to determine that a given binding agent, such as an anti-Notch-1 monoclonal antibody, binds substantially only to Notch-1.

Tumor Cell: A neoplastic cell characterized by increased activity or increased expression of a Notch protein, such as the Notch-1, Notch-2, Notch-3 or Notch-4 protein, relative to the Notch activity or expression in a same tissue type that is not neoplastic. These expression levels can be determined by immunocytochemistry (as most adult tissues have undetectable levels of Notch expression). Examples of tumor types that overexpress Notch-1 include cervical cancer, breast cancer, colon cancer, melanoma, seminoma, lung cancer, and hematopoietic malignancies, such as erythroid leukemia, myeloid leukemia (such as chronic or acute myelogenous leukemia), neuroblastoma and medulloblastoma. An example of a tumor type that overexpresses both Notch-1 and Notch-2 is cervical cancer.

Sequence identity: the similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar are the two sequences.

Methods of alignment of sequences for comparison are well-known in the art. Various programs and alignment algorithms are described in: Smith and Waterman, Adv. Appl. Math. 2:482, 1981; Needleman and Wunsch, J. Mol. Bio. 48:443, 1970; Pearson and Lipman, Methods in Molec. Biology 24: 307-331, 1988; Higgins and Sharp, Gene 73:237-244, 1988; Higgins and Sharp, CABIOS 5:151-153, 1989; Corpet et al., Nucleic Acids Research 16:10881-90, 1988; Huang et al., Computer Applications in BioSciences 8:155-65,1992; and Pearson et al., Methods in Molecular Biology 24:307-31,1994.

The NCBI Basic Local Alignment Search Tool (BLAST) (Altschul et al., J. Mol. Biol. 1215:403-410, 1990) is available from several sources, including the National Center for Biological Information (NBCI, Bethesda, Md.) and on the Internet, for use in connection with the sequence analysis programs blastp, blastn, blastx, tblastn and tblastx. It can be accessed at http://www.ncbi.nlm.nih.gov/BLAST/. A description of how to determine sequence identity using this program is available at http://www.ncbi.nlm.nih.gov/BLAST/blast_help.html.

Another method that can be used to align the at least two sequences is to first align the sequences by hand. Then the number of identical amino acids or nucleotides is counted, and this number divided by the total number of amino acids or nucleotides in the protien or DNA molecule. The resulting number is multiplied by 100, giving the percent identity between the at least two sequences.

Homologs of the Notch proteins are typically characterized by possession of at least 70% sequence identity counted over the full length alignment with the disclosed amino acid sequence using the NCBI Blast 2.0, gapped blastp set to default parameters. Such homologous peptides will more preferably possess at least 75%, more preferably at least 80% and still more preferably at least 90% or 95% sequence identity determined by this method. When less than the entire sequence is being compared for sequence identity, homologs will possess at least 75% and more preferably at least 85% and more preferably still at least 90% or 95% sequence identity over short windows of 10-20 amino acids. Methods for determining sequence identity over such short windows are described at http://www.ncbi.nlm.nih.gov/BLAST/blast_FAQs.html. One of skill in the art will appreciate that these sequence identity ranges are provided for guidance only; it is entirely possible that strongly significant homologs or other variants could be obtained that fall outside of the ranges provided.

The present invention provides not only the peptide homologs that are described above, but also nucleic acid molecules that encode such homologs.

Vector: A nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Purified: The term purified does not require absolute purity; rather, it is intended as a relative term. Thus, for example, a purified peptide preparation is one in which the peptide or protein is more enriched than the peptide or protein is in its natural environment within a cell. Preferably, a preparation is purified such that the protein or peptide represents at least 50% of the total peptide or protein content of the preparation.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Mammal: This term includes both human and non-human mammals. Similarly, the term “subject” includes both human and veterinary subjects.

Animal: Living multicellular vertebrate organisms, a category which includes, for example, mammals and birds.

Mimetic: A molecule (such as an organic chemical compound) that mimics the activity of a protein, such as the activity of the mAbs of Example 4 which enhance differentiation in the presence of a differentiation inducing agent. Peptidomimetic and organomimetic embodiments are within the scope of this term, whereby the three-dimensional arrangement of the chemical constituents of such peptido- and organomimetics mimic the three-dimensional arrangement of the peptide backbone and component amino acid sidechains in the peptide, resulting in such peptido- and organomimetics of the peptides having substantial specific inhibitory activity. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology (ed. Munson, 1995), chapter 102 for a description of techniques used in computer assisted drug design. Example 26 describes other methods which can be used to generate mimetics.

Pharmaceutically acceptable carriers or Pharmaceutical Carrier: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, ethanol, combinations thereof, or the like as a vehicle. The carrier and composition can be sterile, and the formulation suits the mode of administration. For solid compositions (e.g., powder, pill, tablet, or capsule forms), conventional non-toxic solid carriers can include, for example, pharmaceutical grades of mannitol, lactose, starch, sodium saccharine, cellulose, magnesium carbonateor, and magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. The composition can be a liquid solution, suspension, emulsion, tablet, pill, capsule, sustained release formulation, or powder. The composition can be formulated as a suppository, with traditional binders and carriers such as triglycerides.

Tumor: a neoplasm

Neoplasm: abnormal growth of cells

Cancer: malignant neoplasm that has undergone characteristic anaplasia with loss of differentiation, increase rate of growth, invasion of surrounding tissue, and is capable of metastasis.

Malignant: cells which have the properties of anaplasia invasion and metastasis

Normal cells: Non-tumor, non-malignant cells

Cell fate determining function: A cellular function (such as a biochemical pathway) which determines whether a cell will undergo a particular fate, such as growth, differentiation or apoptosis.

### EXAMPLE 1

### Generation of Human Recombinant rh11-12 Antigen

Expression

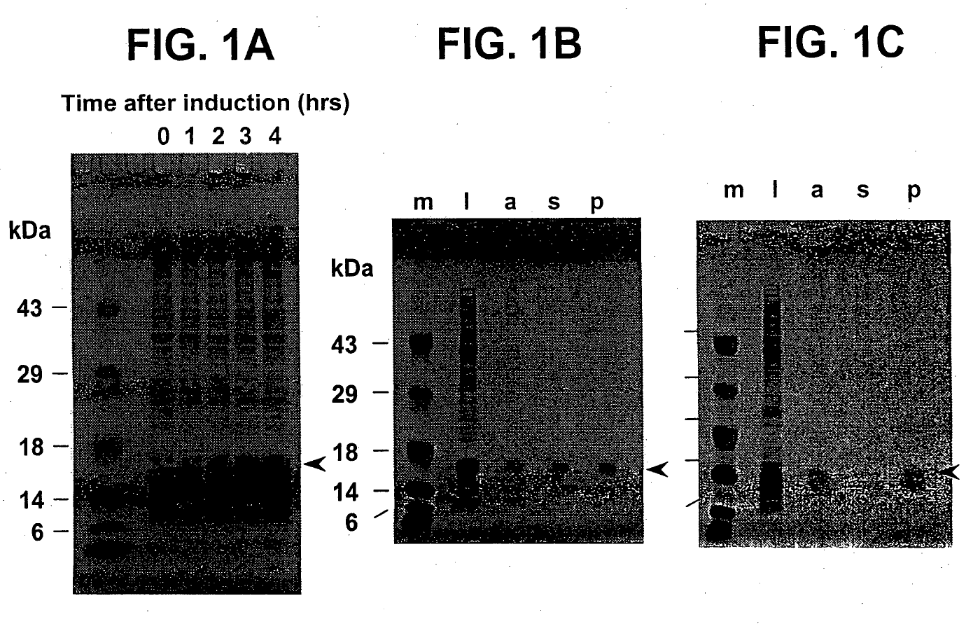

Using PCR, a recombinant cDNA consisting of EGF-repeats 11 and 12 of human Notch-1 (from human thymus cDNA), tagged with a 6-Histidine sequence at the 5′ end was generated. This cDNA was subcloned into expression vector pLD101 (Miele et al., J. Biol. Chem., 1990, 265:6427-35), and the resulting plasmid called pLC11-12. BL21(DE3):pLys-S E. coli transformed with pLC11-12 using heat shock (42° C. for 45 seconds), were grown overnight then diluted 1:400 in fresh LB medium. When the OD₆₀₀ reached 1, production of rh11-12 was induced with 0.45 mM IPTG. Samples were taken at various times. The equivalent of 0.1 ml of culture was lysed directly in SDS-PAGE sample buffer and analyzed on a 4-20% gradient gel, stained with Coomassie. As shown in FIG. 1a, the highest amount of rh11-12 expression was observed after 4 hrs of IPTG treatment. In addition, this expression system produced a soluble, disulfide bonded rh11-12 protein (FIGS. 1B and 1C).

Purification

Bacteria were grown and induced as described above. Cells were harvested 4 hours after IPTG induction and washed once with PBS. Cells were resuspended in SB buffer with protease inhibitors (50 mM NaH₂PO₄, 300 mM NaCl, 1 mM AEBSF, 1 μM leupeptin, 1 μg/ml E-64 and 130 μM bestatin, pH 8.0). This buffer was degassed and equilibrated with nitrogen gas. Pellets were frozen in dry ice/ethanol and thawed twice, then sonicated for 2-3 min in bursts on ice. The lysate was clarified by centrifugation at 14,000×g. The supernatant was equilibrated for 2.5 hours at 4° C. with 8 ml of a 1:1 slurry of NiNTA agarose (Qiagen) with agitation under nitrogen. The slurry was poured into a 1 cm diameter column. The column was washed with at least 20 column volumes of SB buffer, then eluted with a linear gradient of 0 to 0.5 M imidazole. Fractions containing rh11-12 by SDS-PAGE were pooled and concentrated by CentriPrep 3000 filters. The concentrated peak was loaded onto a Sepahdex G-50 superfine column (120 cm).

As shown in FIGS. 1B and 1C, purified rh11-12 eluted in two peaks: aggregates with the void volume(s) and monomer as a “pool 2” peak. Note that aggregates are only visible in non-reduced gels (FIG. 1C), indicating they are disulfide-stabilized.

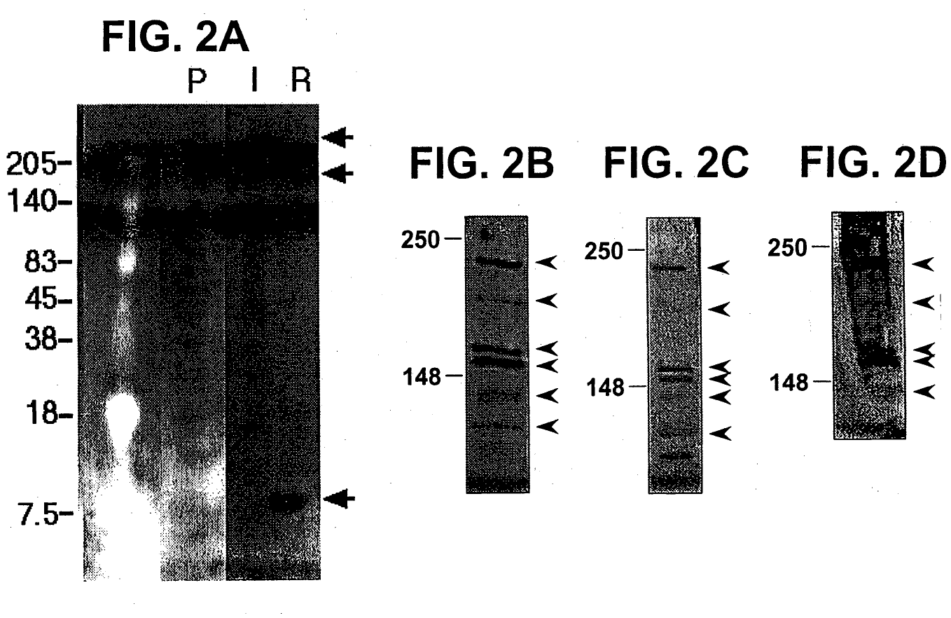

The purified protein contains small amounts of higher aggregates (dimers, trimers, tetramers etc.) which are not visible by SDS-PAGE but are detectable by Western blotting (FIG. 2). This is different than any antigen used in the preparation of other Notch-1 antibodies, since it is native, disulfide-bonded and biologically active (Garcés et al., J. Biol. Chem. 1997, 272:29729-34).

When this recombinant rh11-12 protein was added to 3T3-L1 cells which express Notch-1, it blocked their differentiation (Garcés et al., J. Biol. Chem. 1997, 272:29729-34), suggesting that it can compete with native Notch-1 for its ligand and therefore represents a natural conformational state of the ligand binding region of human Notch-1.

### EXAMPLE 2

### Generation of Notch-1 Polyclonal Antisera

Rabbit antiserum was raised using recombinant rh11-12 (see Example 1) as the antigen, by Cytimmune Inc. (College Park, Md.). Briefly, one NZ rabbit was injected subcutaneously on a monthly basis with purified recombinant rh11-12. To titer the serum, immune and pre-immune sera were compared by ELISA and western blotting with rh11-12. The final antiserum has a titer of 1:10,000 by ELISA.

FIG. 2 shows western blot analysis of human Molt-4 cell lysates. Approximately 2×10⁶ cells were lysed directly in SDS-PAGE sample buffer and analyzed by SDS-PAGE on a 4-20% gradient gel. Western blotting was performed in 10 mM CAPS pH 11 with 10% methanol for 4 h at 0.75 mA. Detection was performed using a Boehringer-Mannheim chemiluminescence kit. As shown in FIG. 2A, this antiserum, lane R, but not the pre-immune serum, lane P, recognized three immunoreactive bands: the Notch-1 preprotein (>207 kDa), the extracellular cleavage product (180 kDa) corresponding to N^{EC} (the mature form of Notch-1 present at the cell surface), and a 190-200 kDa band, that is probably a precursor to N^{EC}. The band at 7.5 kDa is the rh11-12 antigen. These correlate with the expected Notch-1 bands reported in the literature (Blaumueller et al., Perspect. Dev. Neurobiol. 1997, 4:32543). However, these bands are not visible in cells which do not express intact Notch-1 such as SupT1 (Garcés et al., J. Biol. Chem. 1997, 272:29729-34). This polyclonal antibody recognized several species of Notch-1, including human, mouse, and rat, and also recognized Drosophila Notch.

To further analyze the expression of precursor and mature forms of Notch-1, a 4% SDS-PAGE was run to increase the resolution in the high-molecular weight range. Samples were prepared as described above. Sample buffer contained either 10% mercaptoethanol (FIG. 2B), 50 mM dithiothreitol (FIG. 2C) or no reducing agents (FIG. 2D). As shown in FIG. 2b-d, N^{EC} appears as a doublet at 190 and 180 kDa. The faint band at 210 kDa may be a precursor to mature N^{EC}. Smaller forms exist that still may contain EGF-repeats 11 and 12. Finally, the band pattern is not affected by the absence of reducing agents (FIG. 2D), indicating that N^{EC} and the transmembrane subunit N^{TM} are held together by non-covalent interactions.

This polyclonal antibody, when used in the 3T3-L1 system, had the same effect as rh11-12 (see Example 1); it blocked differentiation of adipocytes (Garcés et al. J. Biol. Chem. 1997, 272:29729-34).

### EXAMPLE 3

### Overexpression of Notch-1

The polyclonal Notch-1 antibodies generated in Example 2, were used to monitor the overexpression of Notch-1 in cancer cells. Two human T-cell lymphoblastic leukemia lines (Jurkat in RT-PCR and Molt-4 in Western blotting) are shown side by side with normal human T-cells (obtained from buffy coats provided by the NIH blood bank, and purified by negative selection using kits from R&D Inc.) and fractionated CD4 and CD8 cells (derived from human T cells by further purification with R&D Inc. kits). For RT-PCR, total RNA was extracted from cells using Trizol reagent (Life Technologies). RT-PCR was performed using the Thermostable Reverse Transcriptase RNA PCR kit (Perkin Elmer) according to manufacturer's instructions. Reactions were amplified in a Perkin Elmer 2400 DNA thermal cycler for 40 cycles of denaturation at 94° C. for 1 minute, annealing and extension at 65° C. for 2 minutes. Amplification of mouse primers specific for Notch-1, AATGGTCGAGGACCAGATGG (sense, SEQ ID NO 1), and TTCAGGAGCACAACAGCAGC (antisense, SEQ ID NO 2) generated a product of 431 bp. Negative and positive PCR controls were included in every experiment. For Western blotting, cells were prepared as described in Example 2, using 5% 2-mercaptoethanol as the reducing agent.

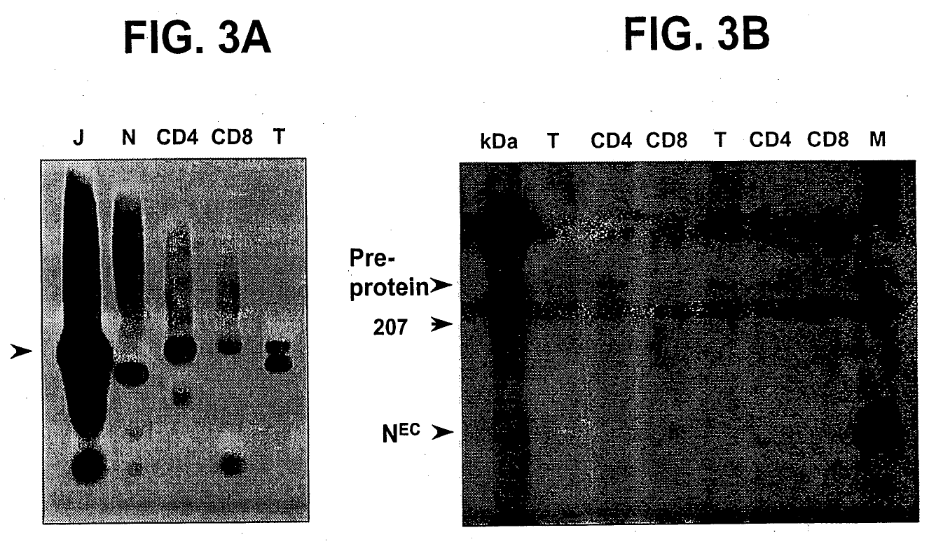

As shown in FIG. 3, the RT-PCR signals from normal cells (lanes N, CD4, CD8) are light, and in the case of the Western blotting (FIG. 3B), barely detectable. In the lymphoblastic leukemia cell lines (lanes J and M), both mRNA (FIG. 3A) and protein (FIG. 3B) appear to be over-expressed by a very large factor (>10 fold).

We also documented overexpression of Notch-1 N^{EC} in human neuroblastoma (SY5Y, DAOY) and medulloblastoma (NGP) cell lines (obtained from NCI), with or without treatment with retinoic acid (RA), a clinically used differentiation-inducing agent. Cells were passaged into fresh medium, at an approximate density of 0.2×10⁶/ml. After 2 days, when cells were logarithmically growing, they were treated with 1×10⁻⁶ M RA for 2 days.

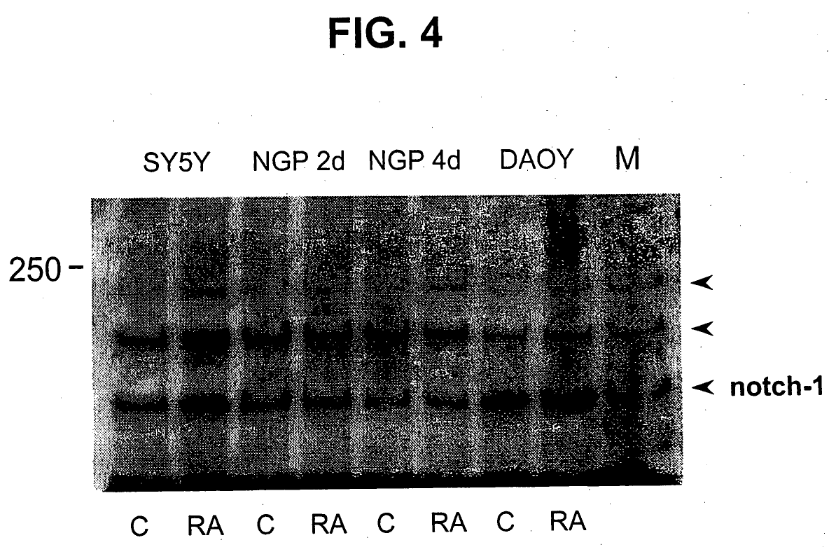

Using western blotting analysis with polyclonal Notch-1 antiserum (see Example 2), the amount of Notch-1 protein expressed in each cell type, in the presence or absence of RA, was determined. As shown in FIG. 4, the level of Notch-1 in the neuronal tumor cell lines (lanes SY5Y, DAOY, NGP) is even higher than in the untreated T-cell leukemia Molt-4 (lane M). The RA treatment also increases the level of Notch-1 expression, especially in neuroblastomas.

### EXAMPLE 4

### Generation of Notch Monoclonal Antibodies

Monoclonal antibodies to any of the Notch proteins can be generated using known techniques (for example see Example 18). The following example, illustrates how anti-Notch-1 antibodies can be produced, but similar techniques can be employed to make a monoclonal antibody against other Notch proteins (for example an antibody that recognizes the ligand-binding region of other Notch proteins, see definition of Notch antibody above) or other regions of the Notch-1 protein. To generate monoclonal anti-Notch-1 antibodies, recombinant rh11-12 (see Example 1) was chemically modified on the N-terminus with N-succinimidyl, S-acetyl-thioacetate (SATA). This was done to introduce a free SH group, after reduction, onto the N-terminal end of rh11-12. In this way, the side chains of rh11-12 amino acids would not be chemically modified and would be free to interact with antigen-presenting MHC molecules after processing. The SATA-modified rh11-12 was conjugated to keyhole limpet hemocyanin (KLH, a common carrier for antigens) and injected into four DBA/2 mice. The mice were boosted every month with unconjugated rh11-12 in incomplete Freund's adjuvant for several months, until antibodies to rh11-12 were detectable by ELISA at good titers in their serum. The animals were sacrificed and spleen cells were fused to murine non-secreting myeloma cells (X63AG8.653) to generate hybridomas in a standard protocol. Fused cells were seeded in several 96-well microplates and the medium was tested by ELISA for rh11-12 antibodies. Positive wells were subjected to two rounds of limiting dilution cloning to obtain single cell-derived (i.e., truly clonal) hybridomas. Clones were screened by ELISA and those having the highest level of signal with rh11-12 were frozen in liquid nitrogen. Approximately 40 IgG and IgM-producing hybridomas were obtained. The three with highest signal were named A6 (IgG2b), C11 (IgG2b) and F3 (IgG2b) and were tested for immunoreactivity to human Notch-1 N^{EC} by several methods. These three clones have been amplified, and characterized, and were deposited to A.T.C.C. (Manassas, Va.) on Mar. 4, 1999 under numbers: HB12654 (A6). HB12656 (C11) and HB12655 (F3).

### EXAMPLE 5

### Characterization of Notch-1 Monoclonal Antibodies

The monoclonal antibodies (mAbs) were analyzed by SDS-PAGE which showed that they were different from one another (data not shown). A6 and F3 are typical immunoglobulins, although slightly different from each other, while C11 seems to lack the disulfide bond joining the two heavy chains and has shorter heavy chains.

Immunoprecipitation and Western Analysis

The mAbs were used in standard immunoprecipitation reactions. Monoclonal antibodies were either pre-bound to protein-A beads or incubated directly with the lysates from Molt-4 cells which overexpress Notch-1 (see FIGS. 3 and 4). To generate cell lysates, cells were incubated in 1% NP40, 50 mM Tris HCl, pH 8.0, high salt (0.5 M NaCl), and a cocktail of protease inhibitors (Boehringer Mannheim #1836145). Cell lysates (4.5×10⁷ cells in 1 ml) were incubated with the mAb (30 ng mAb/μl lysate) for a few hours, captured on protein-A beads with an overnight incubation, washed several times and analyzed by SDS-PAGE followed by Western blotting and detection with the Notch-1 polyclonal antibody (see Example 2). Thus, if the mAbs recognize N^{EC}, they should immunoprecipitate one or more bands that are also recognized by the polyclonal antibody and give a signal in western blotting.

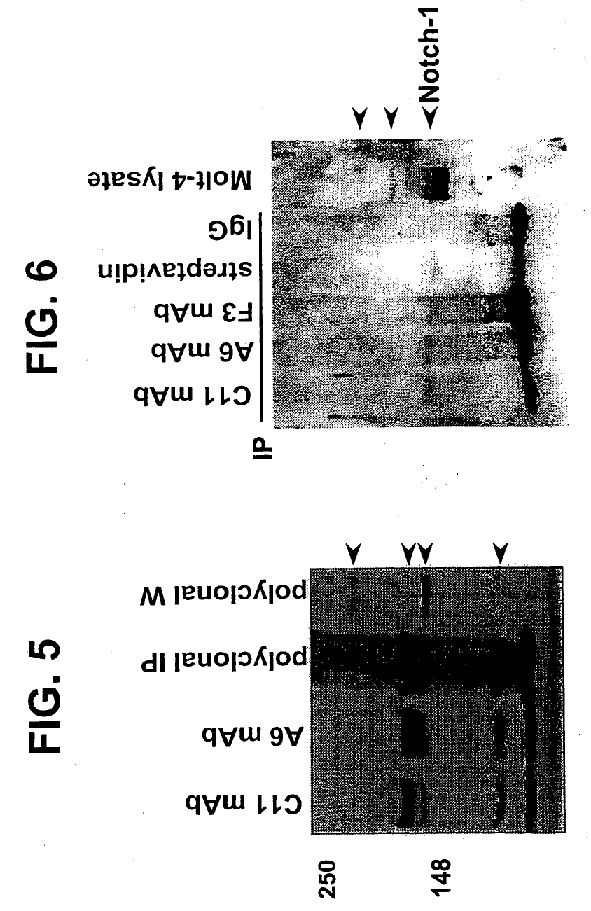

All three mAbs tested, but not a control IgG, immunoprecipitated a doublet of bands corresponding to N^{EC}, but not the Notch-1 pre-protein (FIG. 5). Notch-1 pre-protein is not detected, either because it is not immunoprecipitated by these mAbs or because it is degraded during the incubation. Similar data were obtained with the F3 monoclonal antibody (data not shown).

This data indicates that the mAbs recognize the mature form of human Notch-1. To further confirm this result, intact Molt-4 cells were surface-labeled with biotin, using a Boehringer Manheim kit (#1647652), lysed and immunoprecipitated with A6, F3, C11, control IgG or streptavidin (which binds only biotin and therefore binds all biotinylated proteins on the cell surface). The immunoprecipitated proteins were analyzed using SDS-PAGE and western blotting with the Notch-1 polyclonal antiserum (see Example 2). As shown in FIG. 6, streptavidin and the mAbs, but not control IgG, recognize a 190 kDa band which is recognized by the Notch-1 polyclonal antiserum, and corresponds to the top band in the N^{EC} doublet (Blaumueller et al., Cell 1997, 90:281). This data shows that all three mAbs recognize the form of Notch-1 exposed at the cell surface, and hence the one involved in signaling. This is a significant finding because the majority of Notch-1 is intracellular, and only a small fraction is exposed on the cell membrane.

### EXAMPLE 6

### Detection of Notch In Tumor Cells Using mAbs

This example illustrates how Notch proteins (such as Notch-1) can be detected in tumor cells, to identify tumor cells that express Notch protein and are suitable for treatment with the method of the present invention. To determine if the mAbs generated in Example 5 could recognize native Notch-1, which is overexpressed in certain tumor cells, the mAbs were used to stain sections from a human colon adenomatous polyp which had degenerated into cancer. The carcinoma was formalin fixed, paraffin embedded and cut at 10 μm. Slides containing the tissue sections were incubated in 0.05% pronase (Boehringer Mannheim, 7000 U/g), preheated in 1.47 mM CaCl₂ to 37° C. After a 10 minute incubation, slides were washed in 5 mM EDTA for 5 minutes, rinsed in water, then incubated for 60 minutes in blocking solution (0.5% normal rabbit serum in PBS) in a humidified chamber at room temperature. The slides were then reacted with or without (negative control) mAb at a 1:2 dilution, followed by the ABC Vectastain protocol (Vector Diagnostics) as per manufacturer's instructions. Nuclei were counterstained with hematoxylin. Images of the immunostained tissue were obtained using a Nikon microscope at 200× magnification.

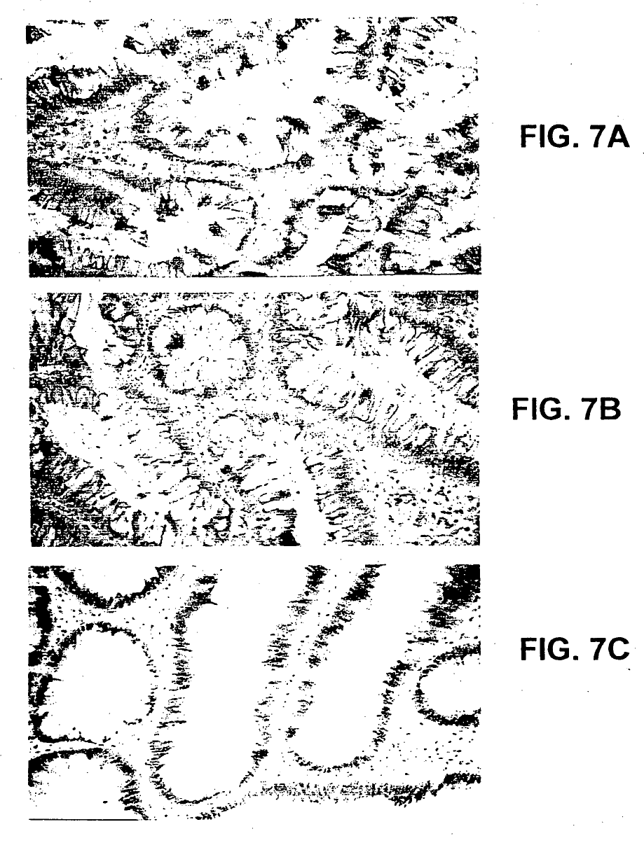

As shown in FIGS. 7A and B, mAbs against Notch-1 EGF repeats 11 and 12 (C11 and F3, respectively) gave a pattern of staining entirely consistent with what had been previously published in tumors overexpressing Notch-1 (Zagouras et al., Proc. Natl. Acad. Sci. USA 1995, 92:6414-8). Similar results were obtained with mAb A6 (data not shown). Note that no staining is observed in the negative control (FIG. 7C). Since these mAbs recognize the form of Notch-1 expressed in human tumors, they have the potential to be used for diagnosing certain tumors, or staging such tumors during their treatment (Examples 19 and 20). Such tumors may include, but are not limited to cervical and prostate cancer, which have been shown to express different levels of Notch-1 during their progression.

### EXAMPLE 7

### Notch Expression is Modulated by HMBA-Induced Cell Differentiation

The regulation of expression of any Notch protein can be monitored following induction of cell differentiation as illustrated in the following example with Notch-1. However, similar procedures can be followed to detect differential regulation of Notch expression, or variations (if any) in the time course of such regulation.

Cell Culture

Murine erythroleukemia Friend cells (MEL), were maintained in RPMI supplemented with 10% (v/v) heat inactivated fetal bovine serum (HyClone) and 10⁻⁵ M β-mercaptoethanol (β-ME). To induce differentiation, logarithmically growing cells were plated at 1×10⁵ cells/ml in medium containing 5 mM HMBA for 120 hours. Cells were passaged to initial density at 72 hours. Cells were assayed for the presence of hemoglobin (Orkin et al., Proc. Natl. Acad. Sci. USA 1975, 72:98-102) by the addition of 1/10 volume of freshly prepared benzidine reagent (0.4% benzidine base, 2% hydrogen peroxide in 12% acetic acid). Benzidine positive cells were counted in modified Neubauer hemocytometers. Each sample was counted by two independent operators, blinded to the cell treatment, and readings were averaged.

Reverse Transcriptase-PCR (RT-PCR)

Total RNA was extracted from cells using Trizol reagent (Life Technologies). RT-PCR was performed using the Thermostable Reverse Transcriptase RNA PCR kit (Perkin Elmer) according to manufacturer's instructions. Reactions were amplified in a Perkin Elmer 2400 DNA thermal cycler for 40 cycles of denaturation at 94° C. for 1 minute, annealing and extension at 65° C. for 2 minutes. Amplification of mouse primers specific for Notch-1, SEQ ID NO 1, and SEQ ID NO 2, generated a product of 431 bp. Primers specific for GAPDH: sense 5′ TCACCACCATGGAGAAGG 3′ (SEQ ID NO 3) and antisense 5′ CAAAGTTGTCATGGATGACC 3′ (SEQ ID NO 4) generated a 200 bp product. Negative and positive PCR controls were included in every experiment.

HMBA Induction

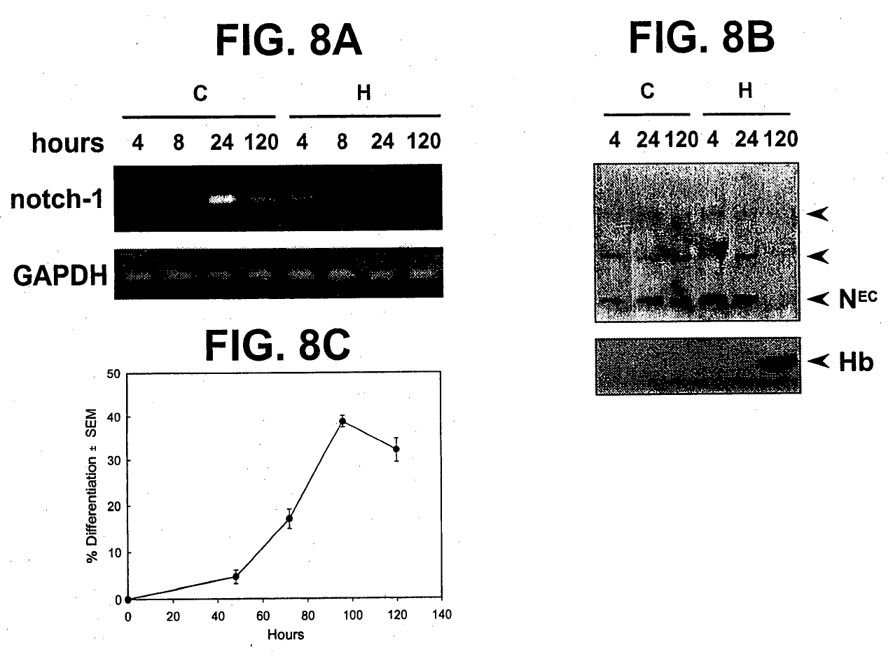

To determine whether Notch-1 expression is regulated during HMBA-induced MEL cell differentiation, Notch-1 mRNA levels were analyzed in MEL cells set up at equal density and maintained in culture for 4, 8, 24 or 120 hours in the absence or presence of HMBA. A representative time course of Notch-1 mRNA levels, determined by RT-PCR analysis, is shown in FIG. 8A. Notch-1 mRNA was observed in MEL cells induced with HMBA for 4 hours, but decreased to undetectable levels in the continued presence of HMBA. In contrast, Notch-1 mRNA was not evident in control cells at 4 and 8 hours, while significant levels were observed at 24 and 120 hours.

Notch-1 steady state protein levels were determined by western blot using the polyclonal antibody to Notch-1 (see Example 2). As shown in FIG. 8B, three immunoreactive bands were detected: the Notch-1 preprotein (>207 kDa), the extracellular cleavage product (180 kDa) corresponding to N^{EC}, and a 190-200 kDa band, probably a precursor to N^{EC}. Notch-1 steady state protein levels reflected the Notch-1 mRNA pattern of expression. In the presence of HMBA (H), Notch-1 protein was reproducibly evident in the early stages of induction, but gradually disappeared becoming essentially undetectable at 120 hours. Significant amounts of hemoglobin (Hb) protein were evident at this time, indicating that erythroid differentiation was taking place. In uninduced MEL cells (C). Notch-1 protein was present at low levels at 4 hours; these levels increased by 24 hours and were maintained throughout the 120 hours. Thus, in the absence of HMBA, Notch-1 accumulates during growth with increasing cell density. HMBA treatment modifies this pattern of expression, causing an early increase in Notch-1 followed by a progressive decline.

The accumulation of differentiated cells, as determined by benzidine staining, was analyzed during the 120 hours of culture with HMBA (FIG. 8C). There was a steady increase in differentiated cells from 48 hours onwards, that generally reached 35-45%, at 120 hours. A slight drop in the number of benzidine-positive cells was observed in some experiments at 120 hours, possibly reflecting a balance between death of previously differentiated cells and differentiation of new cells. Cultures were not continued beyond 120 hours, because at this time Notch-1 was reproducibly undetectable. Expression of Notch-1 was not restored by culturing HMBA-treated cells in fresh medium after day 5 (not shown), demonstrating that commitment to terminal differentiation is accompanied by irreversible loss of Notch-1 expression.

### EXAMPLE 8

### Effect of mAbs on MEL Cell Differentiation

To determine the biological activity of the mAbs generated in Example 4, their effect on cellular differentiation was examined in MEL cells. As described above, there is evidence that downregulating Notch-1, by adding recombinant rh11-12 protein or polyclonal Notch-1 antibodies to 3T3-L1 cells, prevents differentiation (Garcés et al., J. Biol. Chem. 1997, 272:29729-34). In contrast, HMBA induces differentiation in MEL cells (see Example 7). To determine if the mAbs would prevent differentiation like the polyclonal antibodies, their effect on MEL cell differentiation alone, and in the presence of HMBA differentiation agent, was tested.

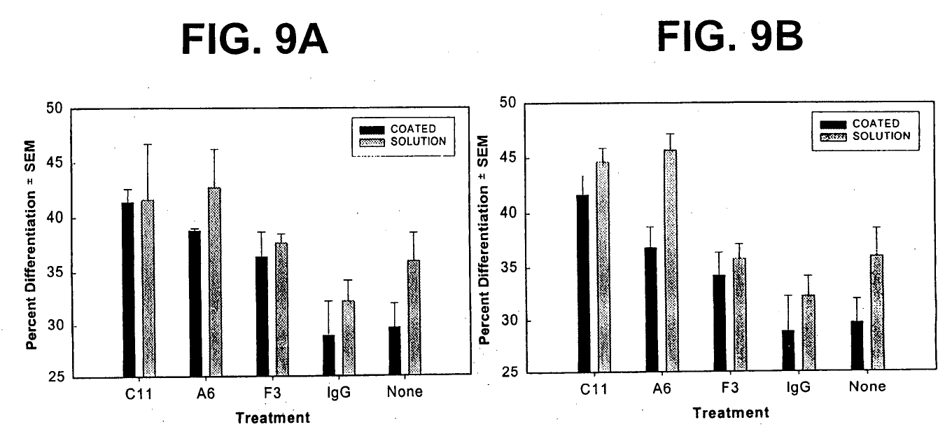

MEL cells were grown as described in Example 7. To examine differentiation, logarithmically growing cells were plated at 1×10⁵ cells/ml in medium containing 5 mM HMBA alone (FIG. 9, none) or in the presence of one of the three mAbs (C11, A6 or F3) or control IgG, for 120 hours. Antibodies were used in solution (10 μg/ml) or coated to the tissue culture plate (1 μg/ml). The mAbs were produced using acites (FIG. 9A) or a hollow fiber bioreactor (FIG. 9B), and then purified by protein A affinity chromatography. Cells were passaged to initial density at 96 hours then assayed for the presence of hemoglobin as described in Example 7.

FIG. 9A shows the results for mAbs that were produced using acites. Unexpectedly, all three mAbs increased differentiation in the presence of HMBA. Although all three mAbs had the same pattern of biological activity, the effect of F3 was weaker. Both A6 and C11 mAbs stimulated differentiation in the presence of HMBA at day four, regardless of whether the antibodies were supplied in solution or coated onto the plate. However, it appears that A6 was better than C11 in solution, and C11 was slightly better than A6 when coated onto a tissue culture dish. In contrast, F3 monoclonal antibody showed significant stimulation only when coated onto the tissue culture plate (compare coated “F3” to coated “none”).

To confirm that the observed effect of the mAbs, to increase cellular differentiation in the presence of HMBA, was not due to contaminants present in the original preparation nor on the manufacturing method of the mAbs, the effect of mAbs produced using a hollow fiber bioreactor, a more common way of manufacturing mAbs for clinical purposes, was tested. Identical results were obtained using this different preparation of mAbs. As shown in FIG. 9B, all three mAbs increased differentiation in the presence of HMBA. A6 and C11 have equivalent potencies in solution, while C11 may be somewhat more potent when coated. In agreement with the earlier results, F3 is the least potent antibody, and appears to have more effect when coated onto the tissue culture dishes. This data indicates that the effect of the mAbs on differentiation in the presence of HMBA does not depend on the manufacture of the mAbs, and it is not an artifact due to contaminants present in mouse ascites fluid.

To determine if the mAbs alone would stimulate differentiation, cells were treated with antibody, in the absence of HMBA. No detectable effect on differentiation was observed (data not shown). This demonstrates that it is the combination of a differentiation-inducing agent, such as HMBA, and an anti-Notch-1 EGF-repeat 11-12 mAb that gives the effect.

When the mAbs were coated onto the tissue-culture plates, there were effects on cell attachment, which varied with the mAbs. F3 and A6 did not affect the morphology of MEL cells, although A6 seemed to induce some attachment, while C11 caused massive cell attachment to the surface. A6 seems to be the best bet for clinical use because its immunoglobulin structure is not atypical (it contains the disulfide bonds joining the two heavy chains, which C11 appears to lack) and it works in solution.

By day 5, the wave of differentiation was over (data not shown), indicating that the effect of these mAbs is to accelerate HMBA-induced differentiation (as shown in Example 7). This terminal differentiation is eventually followed by apoptosis (between 5-7 days). Therefore, the overall effect of the mAbs, when combined with a differentiation inducing agent, would be to increase the rate of differentiation in rumor cells, prior to apoptosis. This treatment is advantageous for patients who cannot tolerate drugs which quickly induce apoptosis. Such patients include the elderly, and post-menopausal women with breast or ovarian cancer.

The mAb effect on HMBA-induced differentiation was unexpected. Based on the published literature and the inventors' observation in the 3T3 cells with the polyclonal serum, an inhibition of differentiation (Garcés et al. J. Biol. Chem. 1997, 272:29729-34) would have been expected. Instead, increased differentiation was observed.

In summary, there are three mAbs to the same antigen, A6, C11 and F3, all of which immunoprecipitate and immunostain tumor cells, but have different degrees of the same biological activity.

### EXAMPLE 9

### Effect of mAbs on Apoptosis

As noted in Example 8, treatment of cells with the monoclonal antibodies (Example 4) alone had no effect on differentiation, in the absence of co-incubation with a differentiation inducing agent. To determine if the same result would occur if cells were treated with the mAbs prior to the differentiation agent, 2×10⁶/ml MEL cells were treated with A6 mAb or IgG2b isotype negative control (Pharmingen) at 20 μg/ml overnight. The following day, cells were washed (to remove unbound antibodies) and resuspended in 5 mM HMBA. After 48 hours, the amount of apoptosis was determined. Cells were washed twice in FACS buffer (PBS/2% FCS/0.02% NaN₂), then incubated with biotin-conjugated anti-mouse IgG2b (Pharmingen) (10 μg /ml) for 30 min at 4° C. Cells were washed twice with FACS buffer, then incubated with streptavidin-FITC (or PE) (Pharmingen) (1:100) for 30 min at 4° C. This allows one to discriminate between cells that have antibody bound (FITC positive), versus those with no antibodies bound (FITC negative). After washing again, apoptosis was determined by FITC-Annexin V/PI staining by flow cytometry using a kit from Pharmingen and a Facscalibur instrument (Becton Dickinson).

Annexin V Binding Assay

Cells undergoing early apoptosis were identified by binding of Annexin-V to membrane phosphatidylserine and assayed using FITC-conjugated Annexin-V (Pharmingen) according to the manufacturer's instructions. After washing, cells were resuspended at 1×10⁶ cells/ml in 1× binding buffer (Pharmingen). Propidium iodide (PI, final concentration 5 μg/ml) and Annexin-V (5 μl) were added to 1×10⁵ cells, incubated for 15 min in the dark at room temperature and analyzed by flow cytometry using a Becton Dickinson FACScan instrument equipped with CellQuest software.

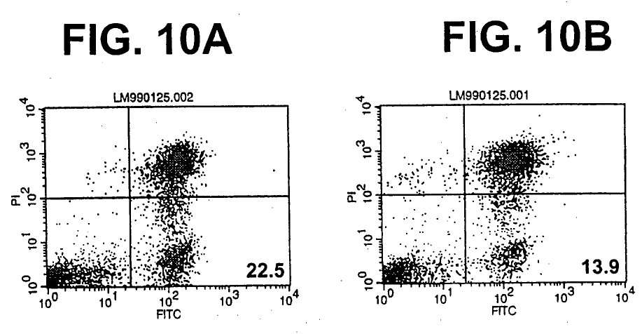

As shown in FIG. 10, treatment of MEL cells with A6 followed by HMBA (FIG. 10A) caused a greater percentage of cells to undergo apoptosis (22.5%) than cells treated with a control antibody followed by HMBA (13.9%) (FIG. 10B). The lower right quadrant in FIG. 10 represents apoptotic cells. Interestingly, this treatment did not induce differentiation. Since this treatment resulted in a 1.6 fold increase in apoptosis, it can be used to enhance apoptosis in chemotherapy patients. In this therapy, patients would be administered an effective amount of mAb first, followed by an effective amount of the chemotherapeutic differentiation inducing agent.

### EXAMPLE 10

### Effect of Notch antisense S-oligos on MEL Cell Differentiation and Apoptosis

As demonstrated above in Examples 8 and 9, antibodies that interfered with Notch activity enhanced both the differentiation and apoptosis of MEL cells in the presence of a differentiation inducing agent. To determine if antisense S-oligos would have a similar effect on cellular differentiation and apoptosis, their biological activity was examined in MEL cells. Although the example illustrates the use of Notch-1 antisense oligonucleotides, antisense can be used to disrupt cellular expression of other Notch proteins.

Antisense Oligonucleotides

Phosphorothioate oligonucleotides (S-oligos) were synthesized in the conventional manner (Agrawal and Zhao, Curr. Opin. Chem. Biol. 519-28, 1998), and sequences for the S-oligos were as follows:

EGF repeat region:

| | | |
|---|---|---|
| sense | GCTGTCTCAACGGTGGTACATGC; | (SEQ ID NO 5) |
| antisense | GCATGTACCACCGTTGAGACAGC; | (SEQ ID NO 6) |

Lin/Notch region:

| | | |
|---|---|---|
| sense | CCTGGAAGAACTGCACGCAGTCT; | (SEQ ID NO 7) |
| antisense | AGACTGCGTGCAGTTCTTCCAGG, | (SEQ ID NO 8) |
| scrambled | GGACCTTCTTGACGTGCGTCAGA; | (SEQ ID NO 9) |

Ankyrin region:

| | | |
|---|---|---|
| sense | CAGCTTGCACAACCAGACAGACC; | (SEQ ID NO 10) |
| antisense | GGTCTGTCTGGTTGTGCAA-GCTG, | (SEQ ID NO 11) |
| scrambled | TGCACGGTTCTGGTTGCGTGTGA. | (SEQ ID NO 12) |

Antisense molecules can be generated for other Notch molecules, for example Notch-2, Notch-3, or Notch-4, as well as for Notch molecules in other species. To design an antisense oligonucleotide, the mRNA sequence from the desired molecule is examined. Regions of the sequence containing multiple repeats, such as TTTTTTTT, are not as desirable because they will lack specificity. Several different regions can be chosen. Of those, oligos are selected by the following characteristics: ones having the best conformation in solution; ones optimized for hybridization characteristics; and one having less potential to form secondary structures. Antisense molecules having a propensity to generate secondary structures are less desirable.

Results

S-oligos corresponding to these regions of chick Notch-1 were described previously by Austin et al. (Development 1995, 121:3637-3650). For S-oligo treatment, MEL cells were induced to differentiate as described in Example 7, with the following modifications. Cells were plated in 96 well plates and S-oligos were added with the medium to a final concentration of 25 μM. Preliminary dose ranging experiments established this as the optimal concentration under the experimental conditions. Both the medium and S-oligos were replaced on day three. At least three independent batches of S-oligos were used. A scrambled control S-oligo was used to rule out artifactual effects due to base composition, and had similar effects to sense S-oligos (data not shown). Comparison to the available sequences in the Genbank database indicated that sequences of the Notch-1 antisense S-oligos were specific to Notch-1.

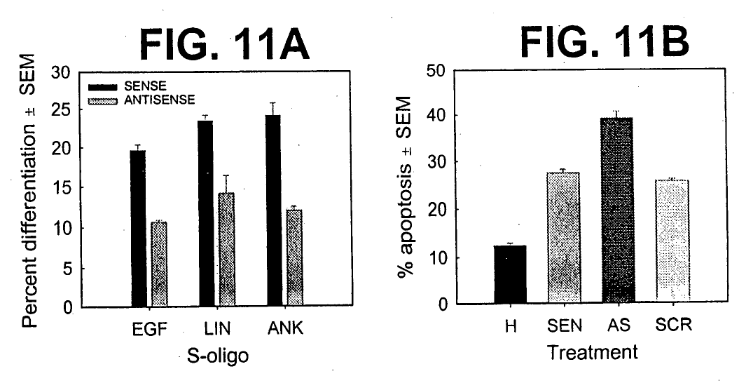

To show that Notch-1 plays a role in HMBA-induced differentiation of MEL cells, Notch-1 antisense S-oligos were added to cells to deregulate expression of the protein. MEL cells were maintained in culture with HMBA for 120 hours, with the addition of the S-oligos at time 0. Benzidine-positive cells were scored at 120 hours. The percentage of benzidine-positive cells was reproducibly decreased by approximately half in the presence of antisense as compared to sense Notch-1 S-oligos (FIG. 11A). This inhibition was seen with each of the three Notch-1 antisense S-oligos. These results show that that this was a true antisense effect specific for Notch-1. When Notch-1 antisense S-oligos were added at day three instead of time 0, differentiation was not inhibited (data not shown). Hence Notch-1 expression is important during the early stages of HMBA induced differentiation, in agreement with the results shown in FIG. 8. In particular embodiments of the invention, the cells are exposed to Notch-1 antisense oligos during early stages of induced differentiation, when the effect of the antisense oligos is more pronounced.

The late apoptotic fraction (sub-G1 peak) in cells treated with Lin-12 sense, antisense and scrambled S-oligos in the presence of HMBA was determined by flow cytometry (FIG. 11B). While all S-oligos increased the apoptotic fraction compared to HMBA alone, the antisense S-oligo had a significantly larger effect than either sense (SEN) or scrambled (SCR) controls (p less than 0.001). A parallel decrease in viability (cells within the G1, G2/S or M regions) was observed. The Lin-12 antisense S-oligo, but not the sense or scrambled control, decreased expression of Notch-1 protein by 50-75% at 48 hours in three experiments. No difference in actin levels was observed in cells treated with sense, antisense and scrambled Lin-12 S-oligos. Actin expression was also unaffected by a transfected 1100 bp construct in a plasmid, as discussed below in Example 11. These results strongly indicate that this is a specific antisense effect, and that Notch-1 expression is required during early stages of HMBA induced differentiation.

### EXAMPLE 11

Effect of Antisense Constructs on Notch-1 Expression

The experiments with S-oligos in Example 10 indicated that Notch-1 is necessary for induced differentiation (such as that induced by HMBA), and that decreases in Notch-1 expression may result in increased apoptosis in these cells. To further characterize the possible role of Notch-1 in cell fate determination during induced differentiation in MEL cells, MEL cells were stably transfected with a Notch-1 antisense molecule. Although the example illustrates the use of Notch-1 antisense oligonucleotides, antisense can be used to disrupt cellular expression of other Notch proteins.

Preparation of Notch-1 Antisense Plasmid

The pNotch-AS plasmid was generated by PCR amplification using cDNA fragments coding for mouse Notch-1 (obtained from Vijaya Manohar, CBER). Nucleotides +64-+1164 were amplified using Pfu DNA polymerase (Stratagene) and cloned in antisense orientation into the XhoI and NheI sites of pcDNA 3.1 (InVitrogen). The primers used, including embedded restriction sites, were TTACTCGAGGCAGCTGGCGAGCAGGCATG (sense, SEQ ID NO 13) and TTAGCTAGCCGGACATTCGCAGTAGAAGG (antisense, SEQ ID NO 14). The nucleotide sequence and orientation of the insert were confirmed by dideoxy sequencing using a Sequenase kit (Amersham).

Transfection of MEL Cells

Logarithmically growing MEL cells were pelleted, resuspended in 200 μl RPMI with 10% FCS and 20 μg of pNotch-AS plasmid or pcDNA3.1 vector plus 2 μg of pBABE to confer puromycin resistance (Morgenstern et al., Nucleic Acid Res. 1990, 18:3587-96). Cells were electroporated using a Bio-Rad gene pulser at 250 V and 960° F. Cells were selected in the presence of 0.5 μg/ml puromycin (Sigma) and 700 μg/ml G418 (Life Technologies) to increase the selective pressure for stable transfectants. Individual clones were isolated by limiting dilution.

Cell Growth Analysis of Notch-1 Transfected Clones

Cells were plated at 1×10⁵ cells/ml (no HMBA) or 2×10⁵ cells/ml (HMBA) in the same medium as parental MEL supplemented with 700 μg/ml G418. Cells were passaged at 72 hours as described above. Cells were counted every 24 hours for 120 hours by hemocytometer.

Western Blotting

Cell pellets were solubilized in hot 2×SDS sample buffer containing 10% β-ME and analyzed by 4% SDS/PAGE. Proteins were electroblotted to Immobilon P (Millipore) in 10 mM CAPS with 10% methanol at 0.75 A for 5 hours. Notch-1 proteins were detected using a chemiluminescence Western blotting kit (Boehringer Mannheim) according to manufacturer's instructions. Relative band intensities were determined by the Kodak 1-D analysis software.

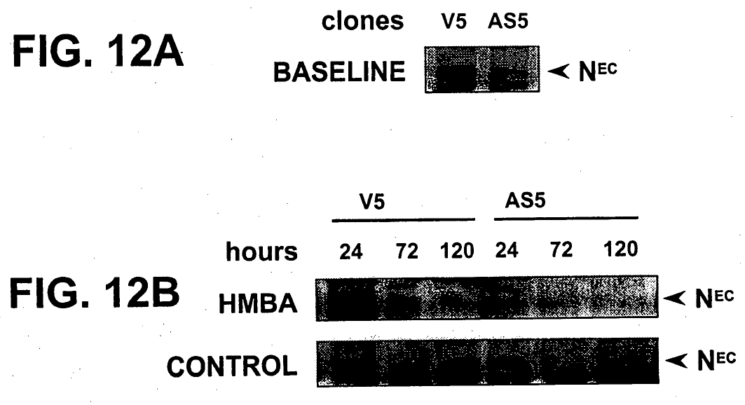

MEL clones were stably transfected with a 1100 bp antisense Notch-1 construct (Notch-1 AS) or with vector alone. Equal amounts of protein (50 μg) isolated from cell extracts of representative Notch-1 AS (AS5) and vector (V5) transfected clones were analyzed by Western blot analysis as shown in FIG. 12.

The antisense construct encompasses the nine N-terminal EGF repeats of the Notch-1 preprotein. This region is less conserved among Notch family members than the rest of the extracellular subunit, and does not include the sequences targeted by the antisense S-oligos. Several individual clones were obtained for each construct and analyzed by Western blot. In all the Notch-1 AS clones used for further experiments, the basal levels of Notch-1 protein were reduced by at least 50% in cells transfected with the Notch-1 AS compared to vector-transfected clones that did not express the Notch-1 AS polynucleotide (FIG. 12A).

Time course experiments were performed with four independent clones transfected with Notch-1 AS (the pNotch-AS pcDNA3.1 plasmid) and three clones transfected with the pcDNA3.1 vector alone. The cells were maintained in culture for 120 hours in the presence or absence of HMBA and Notch-1 protein levels determined by Western blot analyses. Representative blots from two clones are shown in FIG. 12B. Notch-1 protein levels in vector transfected clones reflect the pattern seen in parental MEL maintained in HMBA (see FIG. 8B). In Notch-1 AS clones, the decline in Notch-1 protein levels induced by HMBA was accelerated. Levels of Notch-1 protein in the antisense transfectants appeared to be lower at 24 hours than in the vector-transfected controls and became essentially undetectable by 72 hours.

In the absence of HMBA, the vector-transfected clones exhibited a similar pattern of Notch-1 expression (FIG. 12B) as observed in the parental MEL cells under these conditions (see FIG. 8B). In Notch-1 AS transfected clones, Notch-1 protein is detectable at much lower levels than in vector transfected clones at 24, 72 and 120 hours.

### EXAMPLE 12

### Time Course Experiments

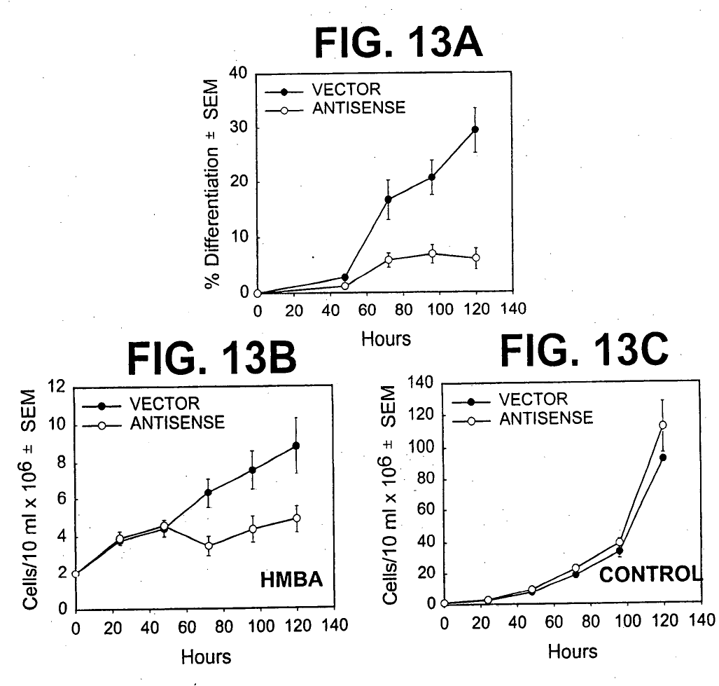

Although the example illustrates the use of Notch-1 antisense oligonucleotides, antisense can be used to disrupt cellular expression of other Notch proteins. Time course experiments were performed with four independent Notch-1 AS clones and three vector clones that did not express Notch-1 AS. The MEL cells were maintained in culture for 120 hours in the presence or absence of HMBA, and the percentage of hemoglobin positive cells in each clone was analyzed at various time points as described in Example 7. In the absence of HMBA, background levels of hemoglobin producing cells (<1%) were observed in both Notch-1 AS and vector transfected cells (data not shown). In the presence of HMBA, differentiation was strongly inhibited in the Notch-1 AS transfected clones compared to the vector transfected cells (FIG. 13A). A marked difference in the percentage of benzidine positive cells between Notch-1 AS versus vector transfected clones was first evident at 72 hours. The percentage of benzidine-positive cells in the AS clones was approximately 6% and did not increase further. In contrast, approximately 17% of the cells were benzidine positive in vector transfected clones at 72 hours and this fraction continued to increase with time reaching approximately 30% at 120 hours.

In MEL cells, proliferation and differentiation are tightly related. To determine if decreases in Notch-1 expression affect proliferation in these cells, the growth kinetics were analyzed for the transfected MEL clones maintained in the presence (FIG. 13B) or absence (FIG. 13C) of HMBA for 120 hours. In these experiments, total cell numbers were determined, without viability corrections. In the presence of HMBA, the Notch-1 AS transfected clones appeared to be essentially growth arrested from 48 hours (FIG. 13B). By contrast, vector-transfected clones showed slow but continued growth throughout the 120 hours (FIG. 13B). In the absence of HMBA, there appeared to be no difference in the growth rates of clones transfected with Notch-1 AS, or vector (FIG. 13C). Growth remained essentially logarithmic up to 120 hours.

### EXAMPLE 13

### Effect of Downregulation of Notch-1 Expression

Experiments using S-oligos indicated that treatment of MEL cells in the presence of HMBA with antisense as opposed to sense Notch-1 S-oligos brought about an increase in apoptosis (see FIG. 11B). To further illustrate this effect, the transfected clones were analyzed by flow cytometry with Annexin V (see EXAMPLE 9) and PI to determine the viability and percentage of cells undergoing apoptosis.

Cell Cycle and Apoptosis Analysis by PI Staining

Parental MEL or transfected clones were synchronized for cell cycle analysis experiments by density arrest (Ryan et al., Mol. Cell Biol. 1993, 13:711-719). At this point, cells were plated at 1×10⁵ cells/ml in medium containing 5 mM HMBA. For the transfected clones, 700 μg/ml G418 was added to the medium. At 16 hours, cells were harvested, fixed in 1% paraformaldehyde for 15 minutes and then 70% ethanol overnight. Cells were resuspended, at constant cell density, in PI solution (50 μg/ml PI (propidum iodide), 0.1% Triton-X-100, 200 μg/ml RNAase A) for 1 hour at room temperature with mixing. In experiments with S-oligos, synchronized cells were plated in 96-well plates with or without S-oligos (25 μM). The DNA content of cells was determined using a Becton Dickinson FACScan flow cytometer. Cells appearing as a sub-G1 peak were scored as apoptotic. In cell cycle analyses, apoptotic and dead cells were gated out and only the viable population was analyzed.

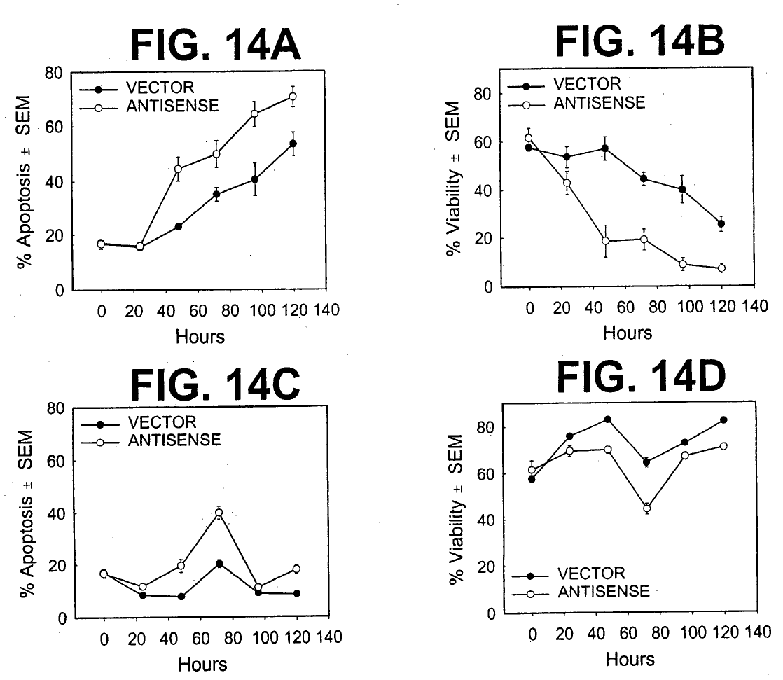

Early apoptotic cells, as determined by the Annexin V positive, PI negative population, were quantitated every 24 hours, in clones maintained in culture for 120 hours in the presence or absence of HMBA. In the presence of HMBA (FIGS. 14A and 14B), both vector and Notch-1 AS transfected clones underwent apoptosis, and the fraction of apoptotic cells increased over time after 24 hours. However, the apoptotic fraction was significantly higher in the Notch-1 AS transfected clones throughout the time course (FIG. 14A), reaching approximately 70% at 120 hours. The percentage of cells undergoing early apoptosis increased dramatically from 24 to 48 hours in the antisense clones compared to vector-transfected cells.

Corresponding changes in viability (PI negative, annexin negative cells) were observed in these clones (FIG. 14B). There was a steady decrease in viability throughout the time course in the presence of HMBA in all clones. However, AS clones showed a much larger drop in viability than vector-transfected clones. In the AS clones, viability dropped linearly from time 0, reaching approximately 18% at 48 hours, compared to 57% in vector transfected clones. At 120 hours, the viability of the AS transfected clones was less than 7% on average, with one clone declining to essentially 0, compared to approximately 30% in the vector transfected clones.

In the absence of HMBA (FIGS. 14C and 14D), apoptosis rates showed a pattern that was roughly parallel to cell density, increasing up to 72 hours. At this time, when cells were passaged into fresh medium at initial density, apoptosis rates dropped (FIG. 14C). This pattern is likely due to growth factor, nutrient or oxygen deprivation in densely growing cultures. While the overall pattern was similar in all clones, the percentage of apoptotic cells in the AS clones was significantly higher than in vector-transfected cells throughout the time course, reaching levels as high as 40% at 72 hours, immediately before cells were passaged into fresh medium. The higher level of apoptosis in AS transfected clones was paralleled by overall lower viability (FIG. 14D). These results show that decreases in Notch-1 expression in MEL cells are associated with increased levels of apoptosis, regardless of the presence of HMBA. In the absence of HMBA, passaging into fresh medium at 72 hours appears to rescue AS cells, since the overall growth curves were not different, hence apoptosis in the absence of HMBA is due to cell growth kinetics. However, to observe massive apoptosis and irreversible cell death, both the differentiation inducing agent and Notch disrupter must be present.

The early G1 lag induced by HMBA is not affected in Notch-1 antisense MEL clones. Treatment of MEL cells with HMBA induces a prolonged G1 phase in the cell cycle immediately subsequent to the first G1 in the presence of the inducer. Metabolic events occurring during the prolonged G1 are known to be important in the decision between continued proliferation or differentiation. To determine if deregulation of Notch-1 expression affects these early cell cycle events, DNA content was analyzed in MEL clones transfected with either Notch-1 AS or vector and induced with HMBA. In these experiments, cells were synchronized in G0/G1 by density arrest, released from synchronization and then maintained in HMBA supplemented medium for 16 hours, and then DNA content in viable cells was determined. Preliminary experiments had shown this to be the time at which a G1 lag was best observed. As shown in Table 1, essentially identical proportions of cells in G1 were observed in Notch-1 AS and vector transfected cells. Similar results were observed in MEL cells treated with Notch-1 sense and antisense S-oligos (data not shown). These experiments indicate that the early G1 lag induced by HMBA is unaffected by reduced levels of Notch-1 protein in this system.

TABLE 1
Cell cycle distribution 16 hours after exposure to HMBA

Percent ± SD

| Cell cycle stage | Vector | AS |
|---|---|---|
| G1 | 52.4 ± 0.8 | 53.2 ± 1.4 |
| G2/S | 26.5 ± 1.3 | 24.4 ± 0.8 |
| M | 21.8 ± 1.9 | 22.9 ± 2.1 |

MEL transfected clones (V5 and AS5) were synchronized by density arrest, and then plated in fresh medium with HMBA. At 16 hours, cells were harvested, stained with PI and analyzed for DNA content. Results are from three independent preparations of each conditions.

### EXAMPLE 14

### Methods of Treatment

Examples 7 and 10-13 illustrate that downregulating the expression of a Notch protein (such as Notch-1) by different antisense strategies, for example targeting different regions in the Notch gene such as the Notch-1 gene), increases apoptosis and decreases differentiation in MEL cells. Examples 8 and 9 illustrate that adding mAbs with HMBA increases differentiation in MEL cells, apparently by downregulating intracellular Notch, which eventually leads to an increase in cellular apoptosis. However, in the absence of differentiation inducer, Notch levels were lowest in freshly passaged cultures and appeared to increase with increasing cell density (Example 7). Conversely, Notch is upregulated early after exposure to a differentiation inducing agent such as HMBA, while commitment to terminal differentiation is associated with the disappearance of Notch, or interference with its function (see Example 7).

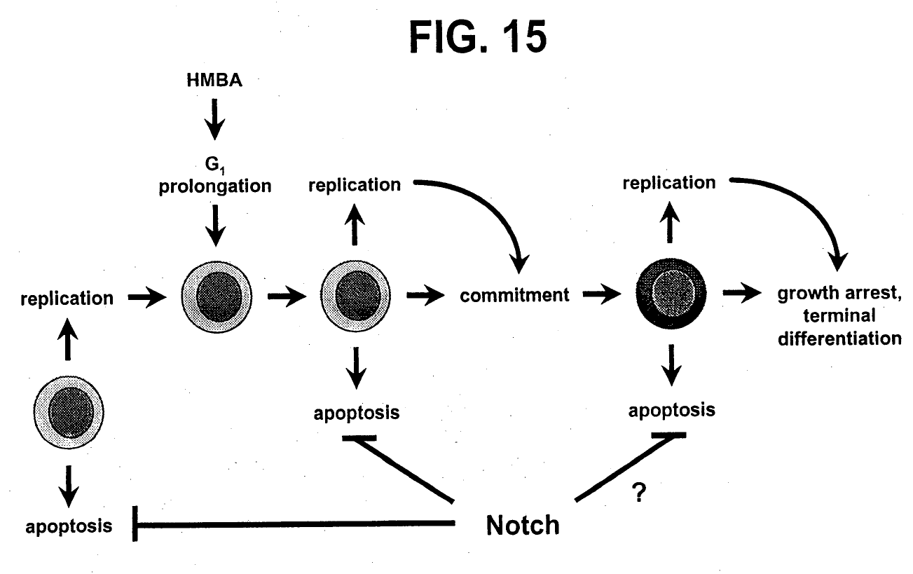

These data indicate that apoptosis susceptibility in MEL cells is controlled by the level of Notch expression, and can also be affected by its timing. Although not wishing to be bound by theory, FIG. 15 summarizes the proposed mechanism by which interference with Notch expression (using antibodies or antisense molecules) in HMBA induced cells induces apoptosis. In cycling MEL cells, Notch controls the apoptosis threshold. During conditions which favor increased apoptosis, such as growth factor deprivation in dense culture, levels of Notch increase, apparently as a protective mechanism. During HMBA-induced differentiation, Notch plays a permissive role, by preventing premature apoptosis of precommitted cells, thus allowing them to continue replicating and produce critical levels of intracellular mediator(s) that result in recruitment to commitment. Notch downregulation may signal commitment to terminal differentiation in MEL cells. When this signal is received prematurely or inappropriately (for example, during the first four hours of differentiation in a MEL cell), the cells appear to become more likely to undergo apoptosis, a common fate of many terminally differentiated cells.

The protective effect of Notch is not specific for apoptosis occurring during HMBA-induced differentiation. Hence Notch modulates common step(s) in the apoptosis pathway in MEL cells rather than a specific signaling cascade which is activated during HMBA treatment. Other differentiation inducing agents can therefore be used, such as: retinoids and derivatives (FDA approved for erythroleukemia, in trials for other malignancies); hybrid polar compounds and derivatives (HMBA being one in which there are phase 2 trials in the treatment of AML and MDS, as reported in Andreef et al., Blood 1992, 80:2604, which provides doses and regimens, and Marks et al., Proc. Nail. Acad. Sci. USA 1994, 91:10251-4 which discloses HMBA analogs); short chain fatty acids (e.g. butyrate) and their derivatives (e.g. tributyrin) and other organic acids (phenylacetate, phenylpropionate); vitamin D derivatives (e.g. vitamin D3); cyclooxygenase inhibitors (COX-1 and 2 inhibitors and specific COX-2 inhibitors) and congeners including natural products with chemopreventive activity (e.g., resveratrol from grapes, which are COX-2 inhibitors, but may have other mechanisms of action as well) and other arachidonate metabolism inhibitors (e.g., phospholipase A2 inhibitors, lipoxygenase inhibitors); ceramides and derivatives (lipids which induce terminal differentiation and apoptosis in some cells); diacylglycerols and derivatives; cyclic nucleotide derivatives (already in clinical use in Europe); hormones, derivatives, antagonists and hormone synthesis inhibitors (e.g., tamoxifen and other anti-estrogens which promote terminal differentiation and inhibit proliferation in breast cancer, androgen antagonists such as finasteride in prostate cancer) and finally, biologics intended to induce terminal differentiation (such as some cytokines, growth factors, peptide hormones, and monoclonal antibodies).

Specific disclosed embodiments use HMBA as the differentiation inducing agent. HMBA is the prototype of a group of hybrid polar compounds, which also includes other hybrid polar compounds shown in Table 2.

TABLE 2
Some Hybrid Polar Compounds

| | *Opt. [ ], | Transformed cells** | | |
|---|---|---|---|---|
| Compound | μM | MEL | HL-60 | HT-29 |
| 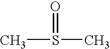 | 280,000 | + | + | + |
| 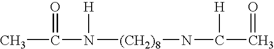 | 5,000 | + | + | + |
| 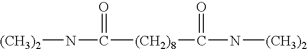 | 5,000 | + | + | + |
| 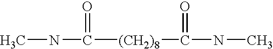 | 5,000 | + | + | + |
| 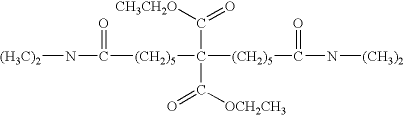 | 200-600 | + | + | + |
| 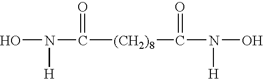 | 10-60 | + | + | + |
| 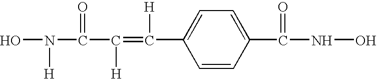 | 10-60 | + | + | + |

*Optimal concentration, in μM
**+, Compound induces terminal differentiation of the transformed cells. MEL are murineerythroleukemia cells, HL-60 are human promyelocytic leukemia cells, and HT-29 are human colon cancer cells.

These compounds are called hybrid polar compounds (HPCs) because they have in common two polar groups, which may be separated by an apolar 5- to 6-carbon alkyl (for example an alkane or alkene) chain, and are soluble in both aqueous and organic solvents.

Certain hybrid polar compounds can be categorized into two classes defined by the identifies of their respective polar groups, which are illustrated in Table 3. Suberoylanilide hydroxamic acid (SAHA) and m-carboxycinnamic acid bishydroxamide (CBHA) (Table 3, compounds 4 and 5) bear at least one hydroxyamide in place of the amides found in HMBA and diethyl bis(pentamethylene-N,N-dimethylcarboxamide)malonate (EMBA) (Table 3, compounds 1 and 2). Techniques for screening HPCs (o determine whether they induce cell differentiation are disclosed, for example, in Richon et al., Proc. Nail. Acad. Sci. USA 95:3003-7, 1998. Additional examples of HPCs are also given in Marks et al., Proc. Nail. Acad. Sci. USA 1994 91:10251-2; Richon et al., Proc. Nail. Acad. Sci. 1996, USA 93:5705-8; and U.S. Pat. No. 5,668,179.

TABLE 3
Two Classes of Hybrid Polar Compounds

| | | | Opt. [ ], | HG cells, |
|---|---|---|---|---|
| No | Name | Structure | μM* | %** |
| 1 | HMBA | 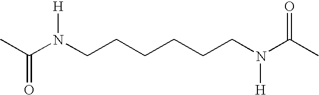 | 5,000 | 95 |
| 2 | EMBA | 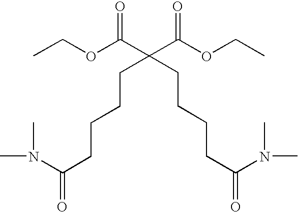 | 300 | 95 |
| 3 | SBHA | 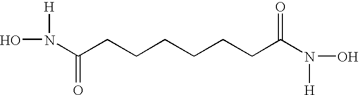 | 30 | 94 |
| 4 | SAHA | 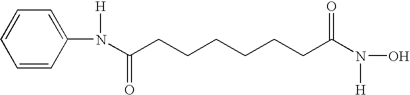 | 2.5 | 68 |
| 5 | CBHA | 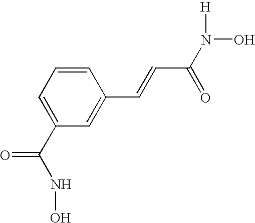 | 4.0 | 73 |
| 6 | TSA | 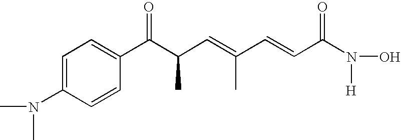 | 0.075 | 80 |
| 7 | 3-Cl- UCHA | 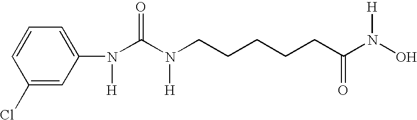 | 4.0 | 32 |

*Optimal concentration, in μM;
**% of cells hemoglobinized

Neoplasms to be evaluated for treatment with the combination treatment of the present invention can include a variety of malignancies and related disorders, such as leukemias, including acute leukemias (such as acute lymphocytic leukemia, acute myelocytic leukemia, and myeloblastic, promyelocytic, myelomonocytic, monocytic and erythroleukemia), chronic leukemias (such as chronic myelocytic (granulocytic) leukemia and chronic lymphocytic leukemia), polycythemia vera, lymphoma, Hodgkin's disease, non-Hodgkin's lymphoma, multiple myeloma, Waldenstrdm's macroglobulinemia, heavy chain disease), as well as solid tumors such as sarcomas and carcinomas, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). Efficacy of the combined therapy of the present invention can be determined by introducing a tumor specimen into one of the assays of the present invention, and determining if apoptosis is increased, or viability is decreased.

In particular embodiments, the tumors are selected for treatment if they exhibit increased Notch protein, such as Notch-1, Notch-2, Notch-3 or Notch-4. In general, overexpression of Notch can be determined by immunocytochemistry (as compared to most adult tissues which have undetectable levels of Notch expression), in-situ hybridization, RT-PCR, and in-situ PCR. Increased Notch expression can be detected by in situ hybridization of cells with a digoxigenin-labeled antisense probe, with detection by alkaline-phosphatase-coupled secondary antibody. For example, the digoxigenin-labeled antisense probe CNOTCH-1 described in Austin et al. (Development 1995, 121:3637-50) can be used to detect Notch-1 expression. Immunologic staining for Notch-1 can also be performed with affinity-purified polyclonal rabbit antibodies against a cytoplasmic domain of human Notch-1, such as T3 (human Notch-1 amino acids 1733-1877), synthesized as a glutathione-5-transferase (GST) fusion protein, with anti-GST antibodies purified from the same animals used as a negative control (Hasserjian et al., Blood 1996, 88:970-6). Alternatively, immunological detection of Notch-1 can be performed with purified polyclonal (see Example 2) or monoclonal (see Example 5) antibodies against the Notch-1 extracellular EGF-repeats 11-12. Antibodies that recognize the ligand-binding)domain of Notch-2, Notch-3 or Notch-4 can also be used.

Tumor cell lines the inventors have found to overexpress Notch-1 include T-cell leukemia lines, myeloid leukemia lines, neuroblastoma, medulloblastoma and colon cancer cell lines. Published information has also indicated that malignancies of the breast, colon, lung (non small cell tumors such as squamous and adenocarcinoma) and cervix (for example carcinoma in situ) exhibit increased expression of human Notch relative to such non-malignant tissue. In addition, both Notch-1 and Notch-2 have been previously shown to be overexpressed in neoplastic lesions of the cervix (Zagouras et al. Proc. Natl. Acad. Sci. USA 1995, 92:6414-18).

Using Notch Antibodies

In an embodiment of the present invention, malignancies in which Notch is overexpressed are treated or prevented by administering an effective amount of the differentiation agent and a monoclonal antibody which recognizes the Notch which is overexpressed. The Notch can include Notch-1, Notch-2, Notch-3 or Notch-4. In specific embodiments, malignancies of the breast, colon, or cervix are treated or prevented by administering an effective amount of the differentiation inducing agent and mAb which recognizes Notch-1 EGF-repeats 11 and 12. The differentiation inducing agent and mAb which recognizes Notch-1 EGF-repeats 11 and 12 may by administered concurrently or separately. If administered separately, the differentiation inducing agent may be administered prior to or simultaneously with the mAb which recognizes Notch-1 EGF-repeats 11 and 12. The differentiation inducing agent is ideally administered at a time that differentiation of the cell can be in progress to such an extent that disruption of Notch expression or function causes the cell to undergo apoptosis, as determined, for example, by the methods set forth in Examples 7 and 8.

Using Antisense Molecules

When Notch levels are prematurely downregulated by various antisense strategies, at a time when differentiation has been induced, the cells tend to enter an apoptotic pathway and abort the differentiation program. The time at which downregulation has this effect can vary between cell types, and can be determined using an assay such as that shown in Examples 7 and 10-13. Notch antisense oligonucleotides for Notch-1, Notch-2, Notch-3 or Notch-4, can be used to disrupt cellular expression of a particular Notch protein.

After performing a round of chemotherapy on a subject, there is a period of time when the tumor cells are actively proliferating. During this time, the subject can be treated with a therapeutically effective amount of Notch antisense. After the Notch antisense has taken affect (Notch levels are downregulated), after 24-48 hours, the subject is treated with a therapeutically effective amount of a differentiation inducing agent. Alternatively, the antisense and the differentiation inducing agent can be administered simultaneously.

Prophylactic Treatements

The treatments of the present invention can also be used prophylactially, for example to inhibit or prevent progression to a neoplastic or malignant state. Such administration is indicated where the combined treatment is shown in assays, as described above, to have utility for treatment or prevention of the disorder. The prophylactic use is indicated in conditions known or suspected of preceding progression to neoplasia or cancer, in particular, where non-neoplastic cell growth consisting of hyperplasia, metaplasia, or most particularly, dysplasia has occurred (for a review of such abnormal growth conditions, see Robbins and Angell, 1976, Basic Pathology, 2d Ed., W.B. Saunders Co., Philadelphia, pp. 68-79). As one example, endometrial hyperplasia often precedes endometrial cancer. Dysplasia is frequently a forerunner of cancer, and is found mainly in the epithelia; it is the most disorderly form of non-neoplastic cell growth, involving a loss in individual cell uniformity and in the architectural orientation of cells. Dysplasia characteristically occurs where there exists chronic irritation or inflammation, and is often found in the cervix, respiratory passages, oral cavity, and gall bladder as an indication that cancer may develop there. In another specific embodiment, an combination therapy of the invention is administered to a human patient to prevent progression to breast, lung, colon, or cervical cancer.

Antibodies which recognize any Notch molecule, such as Notch-1, Notch-2, Notch-3 or Notch-4, may be used to induce differentiation in human tumors, alone or given together with drugs with affect cell differentiation, such as retinoids. In a specific embodiment, the antibody is a monoclonal antibody as described in Example 4 or 18. In one non-cancerous cell line, 3T3 cells, one of the mAbs inhibits terminal differentiation in vitro (Fuchs et al., unpublished). Since Notch-1 is expressed in a number of cells involved in immune functions (T cells, monocytes, possibly others) one maybe able to increase or decrease the function of immune cells which express Notch-1. Application may include immunostimulatory effects or immunosupressive effects, depending on the mAb used (activating or blocking Notch-1 signaling). These could find uses in tumor immunotherapy, vaccines, or the in the treatment of immune disorders and transplant rejection.

Notch antisense molecules which recognize Notch-1, Notch-2, Notch-3 or Notch-4 may be used to downregulate Notch expression for prophylatic treatments. In tumor cells that upregulate Notch expression, the expression of Notch ligands is also upregulated. This activates Notch in surrounding cells (for example lymphocytes), an causes the immune system to loose the ability to recognize Notch expression on the surface of the tumor cells. The downregulating Notch expression using antisense therapies, can enhance the immunity of the tumor.

### EXAMPLE 15

### Pharmaceutical Compositions and Modes of Administration

Various delivery systems for administering the combined therapy of the present invention are known, and include e.g., encapsulation in liposomes, microparticles, microcapsules, expression by recombinant cells, receptor-mediated endocytosis (see, e.g., Wu and Wu, J. Biol. Chem. 1987, 262:4429-32), and construction of a therapeutic nucleic acid as part of a retroviral or other vector. Methods of introduction include, but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, the pharmaceutical compositions may be introduced into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment, for example, by local infusion during surgery, topical application, e.g., in conjunction with a wound dressing after surgery, by injection, through a catheter, by a suppository or an implant, such as a porous, non-porous, or gelatinous material, including membranes, such as silastic membranes, or fibers. In one embodiment, administration can be by direct injection at the site (or former site) of a malignant tumor or neoplastic or pre-neoplastic tissue. In a specific embodiment, administration is performed directly into a Notch-expressing cell by linkage of the agent or molecule to a Delta (or other toporythmic) protein or portion thereof capable of mediating binding to Notch. Contact of a Notch-expressing cell with the linked agent or molecule results in binding via the Delta portion to Notch on the surface of the cell, followed by uptake into the Notch-expressing cell.

The use of liposomes as a delivery vehicle is one delivery method of interest. The liposomes fuse with the target site and deliver the contents of the lumen intracellularly. The liposomes are maintained in contact with the target cells for a sufficient time for fusion to occur, using various means to maintain contact, such as isolation and binding agents. Liposomes may be prepared with purified proteins or peptides that mediate fusion of membranes, such as Sendai virus or influenza virus. The lipids may be any useful combination of known liposome forming lipids, including cationic lipids, such as phosphatidylcholine. Other potential lipids include neutral lipids, such as cholesterol, phosphatidyl serine, phosphatidyl glycerol, and the like. For preparing the liposomes, the procedure described by Kato et al. (J. Biol. Chem. 1991, 266:3361) may be used.

The present invention also provides pharmaceutical compositions which include a therapeutically effective amount of the differentiation-inducing agent and/or Notch disrupting agent, and a pharmaceutically acceptable carrier or excipient.

Delivery Systems

Such carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule, indicating the quantity of active agent. Where the composition is to be administered by infusion, it can be dispensed with an infusion bottle containing sterile pharmaceutical grade water or saline.

The compositions can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, and procaine. The amount of the inducing agent and disrupting agent that will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

Suppositories generally contain active ingredient in the range of 0.5% to 10% by weight; oral formulations preferably contain 10% to 95% of the active ingredients. The invention also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

The pharmaceutical compositions or methods of treatment may be administered in combination with other therapeutic treatments, such as other antineoplastic therapies.

Administration of Nucleic Acid Molecules

In an embodiment in which an analog of a Notch intracellular signal-transducing domain is employed to inhibit Notch signal transduction, the analog is preferably delivered intracellularly (e.g., by expression from a nucleic acid vector, or by linkage to a Delta protein capable of binding to Notch followed by binding and internalization, or by receptor-mediated mechanisms). In a specific embodiment where the therapeutic molecule is a nucleic acid encoding an antisense oligonucleotide, administration may be achieved by an appropriate nucleic acid expression vector which is administered so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 1991, 88:1864-8), etc. Alternatively, the nucleic acid can be introduced intracellularly and incorporated within host cell DNA for expression, by homologous recombination.

The specific vector disclosed in Examples 10-13 pcDNA, is an example of a method of introducing the foreign cDNA into a cell under the control of a strong viral promoter (CMV) to drive the expression. However, other vectors can be used. Other retroviral vectors (such as pRETRO-ON, Clontech), also use this promoter but have the advantages of entering cells without any transfection aid, integrating into the genome of target cells ONLY when the target cell is dividing (as cancer cells do, especially during first remissions after chemotherapy) and they are regulated. It is also possible to turn on the antisense expression by administering tetracycline when these plasmids are used. Hence these plasmids can be allowed to tranfect the cells, then administer a course of tetracycline with a course of chemotherapy to achieve better cytotoxicity.

Other plasmid vectors, such as pMAM-neo (also from Clontech) or pMSG (Pharmacia) use the MMTV-LTR promoter (which can be regulated with steroids) or the SV10 late promoter (pSVL, Pharmacia) or metallothionein—responsive promoter (pBPV, Pharmacia) and other viral vectors, including retroviruses. Examples of other viral vectors include adenqvirus, AAV (adeno-associated virus), recombinant HSV, poxyirases (vaccinia) and recombinant lentivirus (such as HIV). All these vectors achieve the basic goal of delivering into the target cell the cDNA sequence and control elements needed for transcription. The present invention includes all forms of antisense delivery, including synthetic oligos, naked DNA, plasmid and viral, integrated into the genome or not, which work through an antisense mechanism or its closely related antisense ribozyme mechanism (an antisense that also cleaves its target).

Administration of Antibodies

In an embodiment where the therapeutic molecule is an antibody, specifically a mAb that recognizes a Notch protein, combined with a differentiation-inducing agent, administration may be achieved by direct injection, or by use of microparticle bombardment (e.g., a gene gun; Biolistic, Dupont), or coating with lipids or cell-surface receptors or transfecting agents.

The present invention also provides pharmaceutical compositions which include a therapeutically effective amount of the inducing agent with an mAb that recognizes Notch-1 EGF-repeats 11 and 12, and a pharmaceutically acceptable carrier or excipient.

### EXAMPLE 16

### Disruption of Notch Expression

Antisense Disruption

One of the approaches to disrupting Notch function or expression is to use antisense oligonucleotides. The preparation of Notch antisense molecules has been discussed extensively in the scientific literature. For example, oligonucleotides were designed against the mammalian EGF, lin12/Notch and cdc10/ankyrin repeat regions in Austin et al. (Development 1995, 121:3637-50). An antisense oligonucleotide was prepared against six intracellular ankyrin repeats in Garcés et al. (J. Biol. Chem. 1997, 272:29729-34), where the antisense transfected cells displayed inhibition of Notch protein expression, and they lost their ability to undergo differentiation. Techniques for producing Notch antisense oligos, and their effect as Notch disrupting agents, are known.

Generally, the term “antisense” refers to a nucleic acid capable of hybridizing to a portion of a Notch RNA (preferably mRNA) by virtue of some sequence complementarity. The antisense nucleic acids of the invention can be oligonucleotides that are double-stranded or single-stranded, RNA or DNA or a modification or derivative thereof, which can be directly administered to a cell, or which can be produced intracellularly by transcription of exogenous, introduced sequences.

The Notch antisense nucleic acids are polynucleotides, and are preferably oligonucleotides (ranging from 6 to about 100 oligonucleotides). In specific aspects, the oligonucleotide is at least 10, 15, or 100 nucleotides, or a polynucleotide of at least 200 nucleotides. The antisense nucleic acids may be much longer constructs, such as the 1100 bp construct introduced into the plasmid of Example 10. The nucleotides can be DNA or RNA or chimeric mixtures or derivatives or modified versions thereof, single-stranded or double-stranded. The nucleotide can be modified at the base moiety, sugar moiety, or phosphate backbone, and may include other appending groups such as peptides, or agents facilitating transport across the cell membrane (see, e.g., Letsinger et al., Proc. Natl. Acad. Sci. USA 1989, 86:6553-6; Lemaitre et al., Proc. Natl. Acad. Sci. USA 1987, 84:648-52; PCT Publication No. WO 88/09810) or blood-brain barrier (see, e.g., PCT Publication No. WO 89/10134), hybridization triggered cleavage agents (see, e.g., Krol et al., BioTechniques 1988, 6:958-76) or intercalating agents (see, e.g., Zon, Pharm. Res. 1988, 5:539-49).

In a particular aspect of the invention, a Notch antisense polynucleotide (including oligonucleotides) is provided, preferably of single-stranded DNA. The Notch antisense polynucleotide may recognize Notch-1, Notch-2, Notch-3 or Notch-4. In a more particular aspect, such a nucleotide comprises a sequence antisense to the sequence encoding the EGF repeat region, the lin/Notch region, or the ankyrin region, for example of human Notch. The antisense polynucleotide may be modified at any position on its structure with substituents generally known in the art. For example, a modified base moiety may be 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N⁻6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine.

In another embodiment, the polynucleotide includes at least one modified sugar moiety such as arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In yet another embodiment, the polynucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 1987, 15:6625-41). The oligonucleotide may be conjugated to another molecule, e.g., a peptide, hybridization triggered cross-linking agent, transport agent, or hybridization-triggered cleavage agent. Oligonucleotides may include a targeting moiety that enhances uptake of the molecule by tumor cells. The targeting moiety may be a specific binding molecule, such as an antibody or fragment thereof that recognizes a molecule present on the surface of the tumor cell.

As an alternative to antisense inhibitors, catalytic nucleic acid compounds, such as ribozymes or anti-sense conjugates, may be used to inhibit gene expression. Ribozymes may be synthesized and administered to the subject, or may be encoded on an expression vector, from which the ribozyme is synthesized in the targeted cell (as in PCT publication WO 9523225, and Beigelman et al. Nucl. Acids Res. 1995, 23:443442). Examples of oligonucleotides with catalytic activity are described in WO 9506764. Conjugates of antisense with a metal complex, e.g. terpyridylCu (II), capable of mediating mRNA hydrolysis, are described in Bashkin et al., Appl. Biochem Biotechnol. 1995, 54:43-56.

Polynucleotides of the invention may be synthesized by standard methods known in the art, for example by use of an automated DNA synthesizer (Biosearch, Applied Biosystems, etc.). As examples, phosphorothioate oligos may be synthesized by the method of Stein et al. (Nucl. Acids Res. 1998, 16:3209), methylphosphonate oligos can be prepared by use of controlled pore glass polymer supports (Sarin et al., 1988, Proc. Natl. Acad. Sci. USA 85:7448-51). In a specific embodiment, the Notch antisense oligonucleotide comprises catalytic RNA, or a ribozyme (see PCT International Publication WO 90/11364, Sarver et al., Science 1990, 247:1222-5). In another embodiment, the oligonucleotide is a 2′-0-methylribonucleotide (Inoue et al., Nucl. Acids Res. 1987, 15:6131-48), or a chimeric RNA-DNA analogue (Inoue et al., FEBS Lett. 1987, 215:327-330).

The antisense polynucleic acids of the invention comprise a sequence complementary to at least a portion of an RNA transcript of a Notch gene, preferably a human Notch gene. However, absolute complementarity, although preferred, is not required. A sequence may be complementary to at least a portion of an RNA, meaning a sequence having sufficient complementarily to be able to hybridize with the RNA, forming a stable duplex; in the case of double-stranded Notch antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a Notch RNA it may contain and still form a stable duplex (or triplex, as the case may be). One skilled in the art can ascertain a tolerable degree of mismatch by use of standard procedures to determine the melting point of the hybridized complex.

The relative ability of polynucleotides (such as oligonucleotides) to bind to complementary strands is compared by determining the melting temperature of a hybridization complex of the poly/oligonucleotide and its complementary strand. The melting temperature (Tm), a characteristic physical property of double helices, denotes the temperature in degrees Centigrade at which 50% helical versus coiled (unhybridized) forms are present. Base stacking, which occurs during hybridization, is accompanied by a reduction in UV absorption (hypochromicity). A reduction in UV absorption indicates a higher Tm. The higher the Tm the greater the strength of the binding of the hybridized strands. As close to optimal fidelity of base pairing as possible achieves optimal hybridization of a poly/oligonucleotide to its target RNA.

The amount of Notch antisense nucleic acid which will be effective in the treatment of a particular disorder or condition will depend on the nature of the disorder or condition, and can be determined by standard clinical techniques. In a specific embodiment, pharmaceutical compositions comprising Notch antisense nucleic acids are administered via liposomes, microparticles, or microcapsules. In various embodiments of the invention, it may be useful to use such compositions to achieve sustained release of the Notch antisense nucleic acids. In a specific embodiment, it may be desirable to utilize liposomes targeted via antibodies to specific identifiable tumor antigens (Leonetti et al. Proc. Natl. Acad. Sci. USA 1990, 87:2448-51; Renneisen al. J. Biol. Chem. 1990, 265:1633742).

Disruption Using Antibodies

One of the Notch disrupting agents of the invention can be a specific binding agent, such as an antibody (or a fragment containing the binding domain of the antibody) directed against a Notch receptor. The antibody can be used to interfere with the function of the Notch receptor, and interrupt the signaling network that is required to avoid apoptosis of the cell following induction of differentiation. The antibody may include the monoclonal antibodies described in Example 4, the antibodies described in Example 18, as well as antibodies that recognize Notch-1, Notch-2, Notch-3 or Notch-4.

### EXAMPLE 17

### Transgenic Animals Expressing Notch Antisense

Animals which express Notch antisense in their cells have also been prepared to further demonstrate the apoptosis inducing effect of interference with Notch function, when combined with induced tumor cell differentiation. Although this example illustrates expression of Notch-1 antisense, analogous techniques can be used for disruption of expression of other Notch proteins, such as Notch-2, Notch-3, and Notch-4.

A pMAMneo plasmid was used which contained the same insert that was used in the pcDNA 3.1 plasmid that was introduced into the MEL cells. This construct expresses the antisense under a MMTV LTR promoter, which has good basal activity and is stimulated by several steroid hormones, such as estradiol, glucocorticoids and androgens. The plasmid was linearized, purified, and microinjected into mouse embryos which were then implanted into surrogate mothers. Pups were screened for the presence of the transgene by PCR on tail snippets. This was done on two separate instances, which generated one positive founder male and one positive founder female containing Notch-1 antisense in their genomes. The founder animals were separately bred into a C57bl6 background for several generations until homozygous positive mice were obtained which bred true (generated all positive litters).

The animals were viable and fertile, with no gross pathological abnormalities, indicating that apoptosis dose not automatically result from the presence of Notch-1 mRNA in normal cells. These mice have decreased levels of CD8 cells in their thymus, confirming that Notch-1 is necessary for CD8 cell differentiation in the thymus, and indicates that the antisense is affecting known Notch-1 functions. Pharmacological stimuli were used to induce apoptosis more readily in thymocyte cells from these mice.

Thymocytes undergo differentiation in the thymus, and normally express Notch-1. Spontaneous apoptosis levels in thymocytes were not increased, but apoptosis and cell death levels were increased about 50% by a pharmacologic dose of dexamethasone, a steroid used for the treatment of T-cell leukemias which induces rapid terminal differentiation and death in thymocytes. Hence expression of Notch-1 antisense per se does not cause apoptotic cell death, but makes a cell undergo apoptosis when it receives an additional stimulus which induces terminal differentiation. The induced apoptosis was less dramatic than that observed in MEL tumor cells, which are p53 negative, even with 10⁻⁶ M dexamethasone. Hence tumor cells (possibly p53 negative tumor cells) appear to be more sensitive to the lack of Notch-1 than normal cells, which may explain why several tumors overexpress Notch-1. Overexpression of Notch-1 may be one of the mechanisms by which increased resistance to apoptosis is gained by tumor cells. If so, Notch-1 antisense therapy may have potential therapeutic uses in tumors overexpressing Notch-1. Since apoptosis promoted by antisense Notch-1 does not appear to require p53, such strategies may also be used in p53-null tumors.

Tumors may be induced in these transgenic mice to determine whether tumors expressing Notch-1 antisense are more readily treated with differentiation inducing agents. Such studies have supported the finding that Notch-1 antisense requires a pharmacologic stimulus to induce cell death.

### EXAMPLE 18

### Production of Antibodies

Other antibodies that enhance differentiation in the presence of a differentiation inducing agent, as was observed for mAbs A6, C11 and F3, can be generated using Notch proteins, fragments, analogs or derivatives as immunogens. The resulting antibodies can be polyclonal, monoclonal, chimeric, single chain, Fab fragments, or from an Fab expression library. In one embodiment, antibodies which specifically recognize either Notch-1, Notch-2, Notch-3 or Notch-4 can be prepared. In a specific embodiment, antibodies specific to EGF repeats 11 and 12 of Notch-1 may be prepared. For example, a Notch antibody which recognizes Notch-2 EGF-like repeats 11 and 12, and when added to cells in the presence of a differentiation inducing agent, enhances differentiation, can be generated as described for Notch-1 in Example 4. In another embodiment, a Notch antibody is an antibody which recognizes the ligand-binding region of Notch-3. In another embodiment, a Notch antibody is an antibody which recognizes the ligand-binding region of Notch-4. The ligand-binding region is an extracellular domain of Notch. To generate the antibodies, the ligand-binding region, or domains thereof, can be recombinantly expressed, for example in bacteria. The resulting recombinant protein or protein fragment is used to generate antibodies which specifically recognize Notch, and when added to cells in the presence of a differentiation inducing agent, enhances differentiation.

Numerous examples of Notch antibodies can be found in the scientific literature. See for example, Garcés et al., J. Biol. Chem. 1997, 272:29729-34, in which antisera was prepared against the EGF-like repeats 11 and 12 of Notch-1, which spanned positions 1231 to 1471 of the human Notch-1 sequence; the external domain of Notch, Kidd et al., Genes Devel. 1989, 3:1113-29; the T3 cytoplasmic domain (human amino acids 1733-1877) and TC (amino acids 2278-2470); and the unconserved regions of the cytoplasmic domains of Notch-1 and Notch-2, encoded by nucleotides 6658-7131 and 6508-6906, respectively (Zagouras et al., Proc. Natl. Acad. Sci. USA 1995, 92:6414-8).

Monoclonal or polyclonal antibodies may be produced to either the normal Notch protein, a functionally normal Notch protein containing conservative substitutions, peptide fragments, or mutant forms of this protein. Optimally, antibodies raised against the Notch protein will specifically detect the Notch protein. That is, antibodies raised against the human Notch protein would recognize and bind the human Notch protein and would not substantially recognize or bind to other proteins found in human cells. The determination that an antibody specifically detects a Notch protein is made by any one of a number of standard immunoassay methods; for instance, the Western blotting technique (Sambrook et al., 1989). To determine that a given antibody preparation specifically detects the Notch protein by Western blotting, total cellular protein is extracted from human cells (for example, a leukemia cell which over expresses Notch-1) and electrophoresed on a sodium dodecyl sulfate-polyacrylamide gel. The proteins are then transferred to a membrane (for example, nitrocellulose) by Western blotting, and the antibody preparation is incubated with the membrane.

After washing the membrane to remove non-specifically bound antibodies, the presence of specifically bound antibodies is detected by the use of an anti-mouse or anti-rabbit antibody conjugated to an enzyme such as alkaline phosphatase; application of the substrate 5-bromo-4-chloro-3-indolyl phosphate/nitro blue tetrazolium results in the production of a dense blue compound by immuno-localized alkaline phosphatase. Antibodies which specifically detect the Notch protein will, by this technique, be shown to bind to the Notch protein band (which will be localized at a given position on the gel determined by its molecular weight). Non-specific binding of the antibody to other proteins may occur and may be detectable as a weak signal on the Western blot. The non-specific nature of this binding will be recognized by one skilled in the art by the weak signal obtained on the Western blot relative to the strong primary signal arising from the specific antibody-Notch protein binding.

Substantially pure Notch protein suitable for use as an immunogen may be isolated from the transfected or transformed cells. Concentration of protein in the final preparation is adjusted, for example, by concentration on an Amicon filter device, to the level of a few micrograms per milliliter. Monoclonal or polyclonal antibody to the protein can then be prepared as follows.

Monoclonal Antibodies

Monoclonal antibody to epitopes of the Notch protein identified and isolated as described can be prepared from murine hybridomas according to the classical method of Kohler and Milstein (Nature 256:495, 1975) or derivative methods thereof. Briefly, a mouse is repetitively inoculated with a few micrograms of the selected protein over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Enzymol. 70:419, 1980), and derivative methods thereof. Selected positive clones can be expanded and their monoclonal antibody product harvested for use. Detailed procedures for monoclonal antibody production are described in Harlow and Lane (Antibodies: A Laboratory Manual, Cold Spring Harbor Laboratory, New York, 1988). In addition, protocols for producing humanized forms of monoclonal antibodies (for therapeutic applications) and fragments of monoclonal antibodies are known in the art.

Polyclonal Antibodies

Polyclonal antiserum containing antibodies to heterogenous epitopes of a single protein can be prepared by immunizing suitable animals with the expressed protein, which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable.

Booster injections can be given at regular intervals, and antiserum harvested when antibody titer thereof, as determined semi-quantitatively, for example, by double immunodiffusion in agar against known concentrations of the antigen, begins to fall. Plateau concentration of antibody is usually in the range of 0.1 to 0.2 mg/ml of serum (about 12 μM). Affinity of the antisera for the antigen is determined by preparing competitive binding curves.

A third approach to raising antibodies against the Notch protein is to use synthetic peptides synthesized on a commercially available peptide synthesizer based upon the predicted amino acid sequence.

Antibodies may be raised against the Notch protein by subcutaneous injection of a DNA vector which expresses the Notch protein into laboratory animals, such as nice. Delivery of the recombinant vector into the animals may be achieved using a hand-held form of the Biolistic system.

Antibody preparations prepared according to these protocols are useful in quantitative immunoassays which determine concentrations of antigen-bearing substances in biological samples; they are also used semi-quantitatively or qualitatively to identify the presence of antigen in a biological sample.

To screen these antibodies for their ability to enhance differentiation in the presence of a differentiation inducing agent, or their ability to enhance apoptosis when administered prior to the differentiation inducing agent, see Examples 8 and 9.

### EXAMPLE 19

### Methods for Diagnosis

The anti-Notch antibodies described in Examples 2, 4 and 18 can be used in diagnostic applications to detect Notch expression in tumors and pre-cancerous lesions. Antibodies which recognize Notch-2, Notch-3 or Notch-4 can also be used for diagnosis. In addition, antibodies can be used to monitor the change in Notch expression during the course of anti-cancer therapies.

As described in the Background of the Invention, several tumors overexpress Notch-1, relative to Notch-1 expression in the same tissue type that is not neoplastic. In addition, some cancers overexpress both Notch-1 and Notch-2. Using methods described in Example 6, anti-Notch antibodies can be used to determine the level of Notch expression in pathology specimens with light microscopy. Similarly, these anti-Notch antibodies can be used to examine pathology specimens prepared using other histological techniques. These specimens can include, but are not limited to: uterine cervical cancers, premalignant lesions in uterine biopsies, Papanicolaou smears (see Example 20), lung cancer cytology and histology, colon cancer cytology and histology, leukemia/lymphoma cytology and histology and other malignancies.

Anti-Notch antibodies can be detected directly or indirectly. To detect antibodies directly, they need to be directly conjugated with a detectable label. Alternatively, the antibody can be detected indirectly, by adding another antibody, which recognizes the anti-Notch antibody, and has a detectable label.

Labeling Antibodies

Anti-Notch antibodies can be conjugated with various labels for their direct detection (see Chapter 9, Harlow and Lane, 1988, incorporated herein by reference). The label, which may include, but is not limited to, a radiolabel, enzyme, fluorescent probe, or biotin, is chosen based on the method of detection available to the user.

Antibodies can be radiolabeled with iodine (¹²⁵I), which yields low-energy gamma and X-ray radiation. Briefly, 10 μg of protein in 25 μl of 0.5 M sodium phosphate (pH 7.50 is placed in a 1.5 ml conical tube. To this, 500 μC of Na¹²⁵I, and 25 μl of 2 mg/ml chloramine T is added and incubated for 60 sec at room temperature. To stop the reaction, 50 μl of chloramine T stop buffer is added (2.4 mg/ml sodium metabisulfite, 10 mg/ml tyrosine, 10% glycerol, 0.1% xylene cyanol in PBS). The iodinated antibody is separated from the iodotyrosine on a gel filtration column.

Alternatively, anti-Notch antibodies can be labeled with biotin, with enzymes such as alkaline phosphatase (AP) or horseradish peroxidase (HRP) or with fluorescent dyes. The method of producing these conjugates is determined by the reactive group on the label added.

Flow Cytometry

Flow cytometric analysis can be used to analyze intact live cells, obtained from blood, aspriates, bone marrow, or other source. Cells are labeled by methods known to those skilled in the art with a fluorescently-conjugated Notch antibody, which recognizes an extracellular epitope of Notch. Such antibodies can include, but are not limited to A6, C11 and F3. The advantage of flow cytometry is that cells do not need to be fixed or permeabilized prior to their analysis. Cells are washed to remove unbound antibody, then resuspended in an appropriate flow cytometry buffer, such as PBS. Flow cytometric analysis will allow one to determine the percent of cells expressing Notch on the cell surface.

Imaging Methods

For frozen biopsied tissue, the frozen sections are thawed at room temperature and fixed with acetone at −200° C. for 5 min. Slides are washed twice in cold PBS for 5 min each, then air-dried. Sections are covered with 20-30 μl of Ab solution (15-45 μg/ml) (diluted in PBS, 2% BSA at 15-50 μg/ml) and incubated at room temperature in humidified chamber for 30 min. Slides are washed three times with cold PBS 5 min each, allowed to air-dry briefly (5 min) before applying 20-30 μl of the second antibody solution (diluted in PBS, 2% BSA at 15-50 μg/ml) and incubated at room temperature in humidified chamber for 30 min. The label on the second antibody may contain a fluorescent probe, enzyme, radiolabel, biotin, or other detectable marker. The slides are washed three times with cold PBS 5 min each then quickly dipped in distilled water, air-dried, and mounted with PBS containing 30% glycerol. Slides can be stored at 4° C. prior to viewing.

For samples prepared for electron microscopy (versus light microscopy), the second antibody is conjugated to gold particles. Tissue is fixed and embedded with epoxy plastics, then cut into very thin sections (^{˜}1-2 μm). The specimen is then applied to a metal grid, which is then incubated in the primary anti-Notch-1 antibody, washed in a buffer containing BSA, then incubated in a secondary antibody conjugated to gold particles (usually 5-20 nm). These gold particles are visualized using electron microscopy methods.

In addition to ex vivo imaging, anti-Notch antibodies can be used for in vivo diagnostic imaging. Since the monoclonal antibodies described in Example 4 (A6, C11 and F3) recognize the extracellular portion of Notch-1, they do not need to penetrate inside cells. Therefore, the cell membrane does not need to be disrupted. This is especially advantageous for in vivo imaging. Similar antibodies which recognize the extracellular portion of Notch-2, Notch-3 or Notch-4 can be generated using the methods described in Examples 4 or 18.

Radiolabeled antibodies can be administered to a patient intravenously. When the antibody comes into contact with a cell overexpressing Notch-1 on the cell surface, the antibody will bind and emit a radioactive signal. This signal can be detected in many ways including a γ-camera, β-imaging, positron emission tomography (PET) or with SPECT (single photon emission computer tomography) which generates 3-dimensional images. In addition, radioimaging can be performed intraoperatively. During surgery, a radiolabeled mAb solution can be placed onto a region of interest (i.e. lymph nodes, region where a tumor was just resected) and allowed sufficient time to bind. After washing away unbound antibody, a hand-held γ-counter can be used to detect tissue which contains increased Notch. That tissue can then be removed during the surgery, instead of taking random samples and waiting for pathology results. This will allow the surgeon to more completely remove a tumor, and more accurately remove cancerous lymph nodes.

### EXAMPLE 20

### Using Anti-Notch Antibodies to Detect Cervical Cancer in Pap Smears

Antibodies that recognize Notch can be used to detect cervical cancer, which overexpresses Notch, in Pap smears. In one embodiment, the antibodies recognize Notch-1 or Notch-2. In another embodiment, the anti-Notch-1 mAbs described in Example 4 may be used to diagnose cervical cancer, which overexpresses Notch-1. Antibodies currently available against Notch-1 successfully diagnose cervical cancers at the syn3 stage as Notch-1 positive. However, mild pre-cancerous lesions, which may develop into cervical cancer, are detected as Notch-1 negative. Therefore, these antibodies cannot be used to diagnose early stages of cervical cancer.

Currently, if ASCUS (atypical squamous cells of undetermined significance) cells are observed in a routine Pap smear, most doctors recommend a biopsy, because there is currently no objective means to determine which of those patients have cervical cancer, and which do not. However, in only 20% of these cases are the atypical cells determined to be syn3/cervical cancer. Since 80% of these women undergo the biopsy unnecessarily, antibodies which can distinguish between ASCUS cells which require biopsies (cancerous) and those that do not (non-cancerous), would eliminate many unnecessary biopsies. Anti-Notch-1 antibodies currently available cannot do this. However, the mAbs described in Example 4 can be used to make this distinction.

Cervical cells obtained by a Pap smear, may be directly spread onto a microscope slide, or cells may be suspended in solution first, then smeared onto a slide (to provide more uniform cellular distribution). The slides would be immunostained using standard methods described in Harlow and Lane (1988, herein incorporated by reference). A biopsy would be recommended for patients with Pap smears that showed positive Notch-1 staining. Alternatively, the mAbs could be used in a secondary screen, if abnormal cells were detected during the initial screening of the Pap smear.

### EXAMPLE 21

### Enrichment of Notch Expressing Cells

Anti-Notch antibodies can be used to select and/or purify cells expressing high amounts of Notch. Such cells may include, but are not limited to: stem cells from many tissues including bone marrow, intestinal or respiratory epithelia, or tumor cells. Such isolated cells could be used for the purpose of isolation, expansion in culture with or without genetic manipulations and reintroduction into patients.

To enrich for Notch-expressing cells, a heterogeneous population of cells is incubated in the presence of an effective amount of Notch antibody, for example the antibodies described in Examples 2, 4, or 18. The antibody preferentially binds to Notch-expressing cells. After washing cells to remove unbound antibody, cells which have bound antibody are selected. To aid in the selection process, the antibody may be directly or indirectly (using secondary antibodies) radiolabeled, fluorescently labeled, or have magnetic beads attached to it (see Example 19). The cells are then exposed to a flow cytometer (in the case where antibody is detected with a fluorescent probe), which identifies and selects only antibody-labeled cells. Alternatively, the cells can be exposed to a magnetic field, wherein the cells labeled with the magnetic beads attach, while the other cells are washed away. It would be obvious to those skilled in the art that other methods that could be used to enrich/select for cells expressing Notch using antibodies.

### EXAMPLE 22

### Immunoconjugates

Antibodies which recognize Notch, such as Notch-1, Notch-2, Notch-3, or Notch-4, can be used to immunotarget drugs or toxins, as a method of cancer therapy. In a particular embodiment, the antibodies recognize Notch-1, such as the antibodies described in Examples 4 and 18.

Anti-Notch antibodies can be conjugated with drugs, toxins or other desired agent. These agents can be conjugated chemically, using a chemical reaction that is dependent upon the functional groups present on the agent to be conjugated. Immunoconjugates can also be generated using photocrosslinking or other methods known to those skilled in the art. As an alternative approach, recombinant DNA technology can be used. For example, if the antibody is to be conjugated to a toxin, the nucleotide sequence coding for the anti-Notch antibody can be operably linked to the nucleotide sequence coding for a bacterial toxin, such as the Pseudomonas exotoxin. This fused sequence is ligated into a bacterial expression vector, which will express the recombinant fusion protein in vitro. The purified fusion protein, or immunoconjugate, can be administered to patients as described in Example 15.

The immunoconjugate would preferentially target cells overexpressing Notch, for example tumor cells, both in vitro and in vivo. This allows one to concentrate the agent into the cells that overexpress Notch, leaving most non-tumor cells intact. For example, since the mAbs of Example 4 recognize an extracellular epitope of Notch-1, they will bind to the outside of the cell, and be taken into the cells, along with the drug, via endocytosis. Although there are normal cells that express Notch-1 in the brain, the immunoconjugates should not reach them because of the blood brain barrier. In addition, thymocytes express Notch-1, but can be renewed if destroyed by the drug. The agents are selected on the basis that they will kill the tumor cells that the antibody targets.

### EXAMPLE 23

### Modulating Stem Cell Differentiation

Antibodies which recognize Notch, such as Notch-1, Notch-2, Notch-3, or Notch-4, may be used to modulate the differentiation of stem cells in vitro, for the purpose of keeping these cells alive outside of the body until a patient is ready to have these cells administered. In a specific embodiment, the antibodies are the anti-Notch-1 mAbs described in Example 4.

This therapy can be used for allogenic or heterologous bone marrow transplants. Bone marrow cells may be collected directly from the hip by performing a bone biopsy of the anterior or posterior iliac crest. Alternatively bone marrow cells may be collected from the blood, subsequent to treating a patient with drugs which mobilize bone marrow cells into the blood, such as GCSF or naprocine. After collecting bone marrow cells, the heterogeneous population of cells is enriched for the bone marrow stem cells, CD34+cells, as described in Example 24. These CD34+cells are incubated in an effective concentration (10-40 μg/ml) of antibody (for example those described in Example 4) which allows the CD34+cells to survive longer outside the body, than if the cells were not incubated with the antibody.

While the CD34+cells are being kept alive outside the body, the patient may receive chemotherapy and/or radiation therapy to kill any remaining bone marrow stem cells. After this procedure, the enriched CD34+cells are administered to the patient. These CD34+cells may or may not have been genetically altered.

### EXAMPLE 24

### Anti-Notch Antibodies For Use in Immunotherapy

Antibodies which recognize Notch, such as Notch-1, Notch-2, Notch-3, Notch-4, may be used to induce differentiation of CD34+cells into dendritic cells, which are antigen (Ag) presenting cells. In a specific embodiment, the antibodies are the monoclonal antibodies of Example 4.

In this method, blood or bone marrow from a patient is collected, for example by aspiration, and then subjected to methods which allow for the enrichment of CD34+cells from the heterogenous population of cells, such as applying the cells to an affinity column (i.e. a Ceprate column). Blood or bone marrow is incubated in the presence of the CD34+antibody, which recognizes a protein found only on the surface of CD34+cells. After washing away unbound antibody, a secondary antibody, for example a biotinylated antibody, which recognizes the CD34+antibody, is added. The cells are washed again to remove unbound secondary antibody and subjected to an affinity column containing avidin. The interaction between the biotin and avidin will retain the cells within the column, while non-CD34+cells are eluted. After washing, the CD34+cells are eluted and collected. This generates a population of cells, enriched for CD34+cells. It is well known to those skilled in the art that other systems besides biotin and avidin can be used.

To induce the CD34+cells to differentiate into dendritic cells, CD34+cells are incubated in an effective amount of antibody (such as those described in Example 4). The amount of antibody added may be in the range of 10 to 40 μg/ml. In addition, cytokines and other nutrients may be added to maintain the health of the cells.

Once differentiation into dendritic cells has occurred, the dendritic cells are given antigen to present. Dendritic cells are characterized by the expression of (can be CD83 on the cell surface, which can be detected using flow cytometric analysis as described above. The antigen may be administered in the form of a peptide, which is incubated with the dendritic cells, which endocytose the peptide. Alternatively, the dendritic cells may be transfected with a gene that translates the desired antigen.

These antigen-presenting dendritic cells can be used for immunotherapy. In this case of cancer immunotherapy, dendritic cells were given an antigen to present. In addition, this method could be used in vaccine therapies. Those of specific interest are those in which induction of an immune response has failed (i.e. expressing an inactive HIV peptide).

In addition, these antigen-presenting dendritic cells can be used to generate vaccines for cancer. For example, there are currently no effective vaccines for HPV, due to the ineffective immunological response by the patient. In an effort to increase this immunological response, dendritic cells which present an inactive HPV peptide can be generated and administered to patients as a vaccine to protect against HPV infection, which often develops into cervical cancer.

### EXAMPLE 25

### Method for Screening for Agents that Enhance Induced Differentiation

Using method described in Examples 7 and 8, one can identify other agents, compounds, or compositions (hereafter referred to as compounds) that enhance the differentiation observed in the presence of a differentiation inducing agent alone. Briefly, MEL cells are cultured with both a differentiation inducing agent and the compound(s) to be screened for, for 4-120 hours. Controls include the compound alone and the differentiation inducing agent alone. Cells are then screened for their level of differentiation by benzidine staining. The amount of compound(s) and/or differentiation inducing agent can be varied, to identify optimal effective concentrations.

### EXAMPLE 26

### Method for Generating Mimetics

Compounds or other molecules which affect normal Notch function, such as compounds which bind to the same site on Notch-1 as the mAbs of Example 4, and also enhance differentiation in the presence of a differentiation inducing agent, can be identified and/or designed. These non-antibody compounds or molecules are known as mimetics, because they mimic the biological activity of the antibody. The following example is described with respect to Notch-1, but similar techniques can be applied to find mimetics that affect the function of other Notch proteins as well.

Crystallography

To identify the amino acids that interact between the mAbs and Notch-1, Notch-1 is co-crystallized in the presence of the mAb. One method that can be used is the hanging drop method. In this method, a concentrated salt, mAb and Notch-1 protein solution is applied to the underside of a lid of a multiwell dish. A range of concentrations may need to be tested. The lid is placed onto the dish, such that the droplet “hangs” from the lid. As the solvent evaporates, a protein crystal is formed, which can be visualized with a microscope. This crystallized structure is then subjected to X-ray diffraction or NMR analysis which allows for the identification of the amino acid residues that are in contact with one another. The amino acids that contact the antibody establish a pharmacophore that can then be used to identify drugs that interact at that same site.

Identification of Drugs

Once these amino acids have been identified, one can screen synthetic drug databases (which can be licensed from several different drug companies), to identify drugs that interact with the same amino acids of Notch-1 that the mAbs interact with. Moreover, structure activity relationships and computer assisted drug design can be performed as described in Remington, The Science and Practice of Pharmacy, Chapter 28.

Designing Synthetic Peptides

In addition, synthetic peptides can be designed from the sequence of the variable region of the mAb Ig. Several different peptides could be generated from this region. This could be done with or without the crystalography data. However, once crystalography data is available, peptides can also be designed that bind better than the mAbs.

The chimeric peptides may be expressed recombinantly, for example in E. coli. The advantage of the synthetic peptides over the mAbs is that they are smaller, and therefore diffuse easier, and are not as likely to be immunogenic. Standard mutagenesis of such peptides can also be performed to identify variant peptides having even greater differentiation or apoptosis inducing activity.

After synthetic drugs or peptides that bind to Notch-1 have been identified, their ability to enhance differentiation in the presence of a differentiation inducing agent, and/or their ability to induce rapid apoptosis when added prior to the differentiation inducing agent, can be tested as described in Examples 8 and 9. Those that are positive would be good candidates for cancer therapies wherein the cancer cells overexpress Notch.

### EXAMPLE 27

### Effect of Notch Antisense in Combination with AntiNeoplastic Agents

To determine if antisense molecules in combination with antineoplastic agents would have a similar effect on cellular differentiation and apoptosis, as observed for antisense molecules and differentiation agents described in Example 10, the biological activity was examined in SY5Y cells. Although the example illustrates the use of Notch antisense oligonucleotides, anti-Notch antibodies can be used to disrupt Notch protein function.

S-oligos were generated for human Notch-1, as described in Example 10 for murine Notch. Sense, antisense and scrambled S-oligos of 3 regions of Notch-1, huEGF 34/35, huLIN 12 and huCDC-2 were used because these correspond closely to the EGF, Lin/notch and ankyrin regions of the mouse S-oligos. The sequences of the S-oligos were as follows:

| | |
|---|---|
| Hu-EGF 34/35: | |
| antisense | |
| GCAGGTACGAGCGTCATTCTCAC; | (SEQ ID NO 15) |
| sense: | |
| GTGAGAATGACGCTCGTACCTGC; | (SEQ ID NO 16) |
| scrambled: | |
| CACTGACGTGCATCCTTGAGACG; | (SEQ ID NO 17) |
| Hu-LIN 12: | |
| antisense | |
| AGACTGCGTGCAGTTCTTCCAGG; | (SEQ ID NO 18) |
| Hu LIN 12 | |
| sense: | |
| CCTGGAAGAACTGCACGCAGTCT; | (SEQ ID NO 19) |
| Hu-LIN 12 | |
| scrambled: | |
| GTCGGTATGTACTGC GCGTACCA; | (SEQ ID NO 20) |
| Hu-CDC2: | |
| antisense | |
| CCTGGTAGATGAAGTCGGAGATG; | (SEQ ID NO 21) |
| Hu-CDC2 | |
| sense: | |
| CATCTCCGACTTCATCTACCAGG; | (SEQ ID NO 22) |
| Hu CDC2 | |
| scrambled: | |
| GTATCGGACGCGGTTAGAATGGA. | (SEQ ID NO 23) |

SY5Y cells, a neuroblastoma cell line (Example 3) were split into 96 well plates and grown in RPMI 1640 with glutamine, 10% FCS and 1 μM RA. At approximately 60% confluence, the phosphorothioate oligonucleotides (S-oligos, antisense, sense and scrambled; see Example 10) at various concentrations (25, 50, 75 and 100 μM) were added. Subsequently, 50 ng/ml of vinblastine (Sigma, St. Louis Mo., V-1377) was added to half of the wells (based on preliminary dose ranging experiments). Cell survival was measured using Promega's CellTiter 96 AQueous assay, which measures viable cells in the well. Paclitaxel (Sigma, St. Louis Mo., T-1912), another member of the of vinca alkaloid antineoplastic agents family, was also tested (at 50 ng/ml) using this same assay. The results of these studies demonstrated that cell survival was decreased to a greater extent in cells treated with both antisense and the antineoplastic agent, than with either alone.

Antineoplastic agents, which are being used in combination with Notch therapy (for example Notch antisense molecules or Notch antibodies), can be clinically used in accepted clinical protocols, for example those given in the Physician's Desk Reference (1999 Ed.) and in Gilman et al. The Pharmacological Basis of Therapeutics, 7^{th} Ed. Section XIII, Chemotherapy of Neoplastic disease by Calabresi and Parks pp 1240-1306, 1985, Macmilian Publishing Co, New York.

In view of the many possible embodiments to which the principles of our invention may be applied, it should be recognized that the illustrated embodiments are only preferred examples of the invention and should not be taken as a limitation on the scope of the invention. Rather, the scope of the invention is in accord with the following claims. We therefore claim as our invention all that comes within the scope and spirit of these claims.

1. A method of inducing apoptosis in a target cell, comprising:
inducing differentiation of the target cell; and
inhibiting a cell fate determining function of a Notch protein in the target cell at a time when the cell is undergoing differentiation, which induces the target cell to undergo apoptosis.

2. The method of claim 1, wherein the target cell is a tumor cell characterized by:
(a) increased expression of the Notch protein; or
(b) increased Notch activity or expression, relative to Notch activity or expression in a same tissue type that is not neoplastic.

3.-4. (canceled)

5. The method of claim 2, wherein the tumor cell is:
(a) selected from the group consisting of cervical cancer, breast cancer, colon cancer, melanoma, seminoma, lung cancer, and hematopoietic malignancy; and
(b) is a tumor cell in a subject.

6. (canceled)

7. The method of claim 1, wherein inducing differentiation of the target cell comprises administering an effective amount of a differentiation inducing agent.

8. The method of claim 7, wherein the differentiation inducing agent comprises an agent selected from the group of retinoids, polar compounds, short chain fatty acids, organic acids, Vitamin D derivatives, cyclooxygenase inhibitors, arachinodate metabolism inhibitors, ceramides, diacylglycerol, cyclic nucleotide derivatives, hormones, hormone antagonists, and biologic promoters of differentiation, and derivatives thereof.

9. The method of claim 8, wherein the agent is a polar hybrid compound.

10. The method of claim 9, wherein the polar hybrid compound is hexamethylene bisacetamide (HMBA).

11. The method of claim 1, wherein inhibiting the cell fate determining function of Notch protein comprises inhibiting expression of Notch protein in the target cell.

12. The method of claim 11, wherein inhibiting expression of Notch protein comprises exposing the cell to an effective amount of an antisense molecule that specifically blocks expression of Notch protein.

13. The method of claim 12, wherein the antisense molecule includes at least six contiguous nucleotides of a sequence that is complementary to at least a portion of an RNA transcript of a Notch gene, and is hybridizable to the RNA transcript.

14.-15. (canceled)

16. The method of claim 13, wherein the antisense molecule comprises at least six contiguous nucleotides from the group consisting of SEQ. ID. NOS. 6, 8, or 11.

17. The method of claim 1, wherein inhibiting the function of Notch protein comprises exposing the cell to a molecule which antagonizes the function of the Notch protein.

18. The method of claim 17, wherein the molecule which antagonizes the function of Notch protein comprises an antibody that specifically binds to Notch, or a portion of the antibody containing a binding domain that specifically binds to Notch.

19. An antibody generated against the human Notch-1 EGF-repeats 11 and 12, that recognizes an extracellular epitope of Notch-1, and that stimulates target cell differentiation in the presence of an effective amount of a differentiation inducing agent.

20. The antibody of claim 19, wherein the antibody is a monoclonal antibody selected from the group consisting of a) a monoclonal antibody secreted by a hybridoma designated A6 having A.T.C.C. Accession No. HB12654; b) a monoclonal antibody secreted by a hybridoma designated C11 having A.T.C.C. Accession No. HB12656; and c) a monoclonal antibody secreted by a hybridoma designated F3 having A.T.C.C. Accession No. HB12655.

21. (canceled)

22. The method of claim 18, wherein the antibody is an antibody against the human Notch-1 EGF-repeats 11 and 12, that recognizes an extracellular epitope of Notch-1, and that stimulates target cell differentiation in the presence of an effective amount of differentiation inducing agent.

23. The method of claim 22, wherein the antibody is a monoclonal antibody selected from the group consisting of a) a monoclonal antibody secreted by a hybridoma designated A6 having A.T.C.C. Accession No. HB12654; b) a monoclonal antibody secreted by a hybridoma designated C11 having A.T.C.C. Accession No. HB12656; and c) a monoclonal antibody secreted by a hybridoma designated F3 having A.T.C.C. Accession No. HB12655.

24.-31. (canceled)

32. The method of claim 1, wherein inhibiting a cell fate determining function of a Notch protein comprises:
administering a therapeutically effective amount of an antibody generated against the human Notch-1 EGF-repeats 11 and 12, that recognizes an extracellular epitope of Notch-1, and that stimulates target cell differentiation in the presence of an effective amount of differentiation inducing agent; and
wherein inducing differentiation of the target cell comprises subsequently administering a therapeutically effective amount of a differentiation inducing agent.

33.-43. (canceled)

44. The method of claim 1, wherein inducing differentiation comprises stimulating terminal differentiation followed by apoptosis.

45.-62. (canceled)

63. A pharmaceutical composition comprising the antibody of claim 19, wherein the antibody is a monoclonal antibody in a therapeutically effective amount sufficient to stimulate target cell differentiation in the presence of a sufficient amount of a differentiation inducing agent.

64. The pharmaceutical composition of claim 63, further comprising:
(a) a therapeutically effective amount of a differentiation inducing agent selected from the group consisting of retinoids, polar compounds, short chain fatty acids, organic acids, Vitamin D derivatives, cyclooxygenase inhibitors, arachinodate metabolism inhibitors, ceramides, diacylglycerol, cyclic nucleotide derivative, hormones, hormone antagonisits, and biologic promotors of differentiation, and derivatives thereof; and
(b) a pharmaceutically acceptable carrier.

65. The pharmaceutical composition of claim 63, wherein the differentiation inducing agent is HMBA.

66. The pharmaceutical composition of claim 63, wherein the monoclonal antibody is the monoclonal antibody secreted by a hybridoma selected from the group consisting of: a) A6 having A.T.C.C. Accession No. HB12654; b) C11 having A.T.C.C. Accession No. HB12656; and c) F3 having A.T.C.C. Accession No. HB12655.

67. (canceled)

68. The antibody of claim 19, wherein the antibody is a monoclonal antibody and the target cell is selected from the group consisting of cervical cancer, breast cancer, colon cancer, melanoma, seminoma, lung cancer, and hematopoietic malignancy.

69. A polyclonal antibody generated against biologically active human Notch-1 EGF-repeats 11 and 12 that recognizes an extracellular epitope of Notch-1 and induces differentiation of a tumor cell that overexpresses Notch-1, such that when differentiation of the tumor cells is induced, exposure of the cell to the polyclonal antibody induces apoptosis of the cell.

70. The polyclonal antibody of claim 69, wherein the biologically active human Notch-1 EGF repeats 11 and 12 is not reduced to cleave a disulfide bond.

71. (canceled)

72. The method of claim 1, further comprising treating the target cell with a therapeutically effective amount of another antineoplastic agent at a time that enhances apoptosis in the target cell.

73. The method of claim 72 wherein the other antineoplastic agent comprises vinca alkaloid.

74. The method of claim 73 wherein the vinca alkaloids are selected from the group consisting of vinblastine, Paclitaxel and vincristine.

75. The method of claim 72, wherein the antineoplastic agent is administered substantially concurrently with the agent administered to inhibit a cell fate determining function of a Notch protein in the target cell at a time when the cell is undergoing differentiation, which induces the target cell to undergo apoptosis.

76-79. (canceled)
